SEQUENCE LISTING (44 sequences)

SEQ ID NO: 1 - protein, 500 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKAAESPDQKDTEGGPKEEESPV

SEQ ID NO: 2 - protein, 478 aa, Homo sapiens
MPPMPSAPPVHPPPDGGWGWIVVGAAFISIGFSYAFPKAVTVFFKEIQQIFHTTYSEIAWISSIMLAVMYAGGPVSSVLVNKYGSRPVVIAGGLLCCLGMVLASFSSSVVQLYLTMGFITGLGLAFNLQPALTIIGKYFYRKRPMANGLAMAGSPVFLSSLAPFNQYLFNTFGWKGSFLILGSLLLNACVAGSLMRPLGPNQTTSKSKNKTGKTEDDSSPKKIKTKKSTWEKVNKYLDFSLFKHRGFLIYLSGNVIMFLGFFAPIIFLAPYAKDQGIDEYSAAFLLSVMAFVDMFARPSVGLIANSKYIRPRIQYFFSFAIMFNGVCHLLCPLAQDYTSLVLYAVFFGLGFGSVSSVLFETLMDLVGAPRFSSAVGLVTIVECGPVLLGPPLAGKLVDLTGEYKYMYMSCGAIVVAASVWLLIGNAINYRLLAKERKEENARQKTRESEPLSKSKHSEDVNVKVSNAQSVTSERETNI

SEQ ID NO: 3 - protein, 504 aa, Homo sapiens [sequence omitted]

SEQ ID NO: 4 - protein, 465 aa, Homo sapiens
MGGAVVDEGPTGVKAPDGGWGWAVLFGCFVITGFSYAFPKAVSVFFKELIQEFGIGYSDTAWISSILLAMLYGTGPLCSVCVNRFGCRPVMLVGGLFASLGMVAASFCRSIIQVYLTTGVITGLGLALNFQPSLIMLNRYFSKRRPMANGLAAAGSPVFLCALSPLGQLLQDRYGWRGGFLILGGLLLNCCVCAALMRPLVVTAQPGSGPPRPSRRLLDLSVFRDRGFVLYAVAASVMVLGLFVPPVFVVSYAKDLGVPDTKAAFLLTILGFIDIFARPAAGFVAGLGKVRPYSVYLFSFSMFFNGLADLAGSTAGDYGGLVVFCIFFGISYGMVGALQFEVLMAIVGTHKFSSAIGLVLLMEAVAVLVGPPSGGKLLDATHVYMYVFILAGAEVLTSSLILLLGNFFCIRKKPKEPQPEVAAAEEEKLHKPPADSGVDLREVEHFLKAEPEKNGEVVHTPETSV

SEQ ID NO: 5 - protein, 494 aa, Rattus norvegicus
MPPAIGGPVGYTPPDGGWGWAVVVGAFISIGFSYAFPKSITVFFKEIEIIFSATTSEVSWISSIMLAVMYAGGPISSILVNKYGSRPVMIAGGCLSGCGLIAASFCNTVQELYFCIGVIGGLGLAFNLNPALTMIGKYFYKKRPLANGLAMAGSPVFLSTLAPLNQAFFGIFGWRGSFLILGGLLLNCCVAGSLMRPIGPQQGKVEKLKSKESLQEAGKSDANTDLIGGSPKGEKLSVFQTVNKFLDLSLFTHRGFLLYLSGNVVMFFGLFTPLVFLSNYGKSKHFSSEKSAFLLSILAFVDMVARPSMGLAANTRWIRPRVQYFFAASVVANGVCHLLAPLSTTYVGFCIYAGVFGFAFGWLSSVLFETLMDLVGPQRFSSAVGLVTIVECCPVLLGPPLLGRLNDMYGDYKYTYWACGVILIIAGLYLFIGMGINYRLVAKEQKAEEKKRDGKEDETSTDVDEKPKKTMKETQSPAPLQNSSGDPAEEESPV

SEQ ID NO: 6 - DNA, 29 bp, Artificial Sequence
  synthetically generated oligonucleotide
ggatccaccatgccaccagcagttggagg

SEQ ID NO: 7 - DNA, 31 bp, Artificial Sequence
  synthetically generated oligonucleotide
gtcgactcagactggactttcctcctccttg

SEQ ID NO: 8 - DNA, 35 bp, Artificial Sequence
  synthetically generated oligonucleotide
tgcatgatcaatgccacctgcgattggcgggccag

SEQ ID NO: 9 - DNA, 30 bp, Artificial Sequence
  synthetically generated oligonucleotide
tgcagctagctcagactgggctctcctcct

SEQ ID NO: 10 - DNA, 30 bp, Artificial Sequence
  synthetically generated oligonucleotide
agctggatccaccatgccaccaatgccaag

SEQ ID NO: 11 - DNA, 34 bp, Artificial Sequence
  synthetically generated oligonucleotide
gactctcgagttaaatgttagtttctctttctga

SEQ ID NO: 12 - DNA, 25 bp, Artificial Sequence
  synthetically generated oligonucleotide
atcaggatccaggcagcgatgggcg

SEQ ID NO: 13 - DNA, 26 bp, Artificial Sequence
  synthetically generated oligonucleotide
gacacggggcccgtgccgtagagcat

SEQ ID NO: 14 - DNA, 24 bp, Artificial Sequence
  synthetically generated oligonucleotide
cggcacgggccccgtgtccagcat

SEQ ID NO: 15 - DNA, 26 bp, Artificial Sequence
  synthetically generated oligonucleotide
aggcccaggcctgtgagcaccccagc

SEQ ID NO: 16 - DNA, 20 bp, Artificial Sequence
  synthetically generated oligonucleotide
gttcccggatctgctgggtt

SEQ ID NO: 17 - DNA, 20 bp, Artificial Sequence
  synthetically generated oligonucleotide
tggagcttccctgggtctaa

SEQ ID NO: 18 - DNA, 33 bp, Artificial Sequence
  synthetically generated oligonucleotide
ccctctgccggccgcctggtggatgcgttgaag

SEQ ID NO: 19 - DNA, 35 bp, Artificial Sequence
  synthetically generated oligonucleotide
gtcaactagtcagacacccaggggatcaactggag

SEQ ID NO: 20 - DNA, 28 bp, Artificial Sequence
  synthetically generated oligonucleotide
tgctcacaggcctgggcctggccctcaa

SEQ ID NO: 21 - DNA, 26 bp, Artificial Sequence
  synthetically generated oligonucleotide
accaggcggccggcagagggcggtcc

SEQ ID NO: 22 - DNA, 25 bp, Artificial Sequence
  synthetically generated oligonucleotide
taggaagaagcccaaagagccacag

SEQ ID NO: 23 - DNA, 35 bp, Artificial Sequence
  synthetically generated oligonucleotide
gacttctagagcccagccactcagacacttgtttc

SEQ ID NO: 24 - DNA, 27 bp, Artificial Sequence
  synthetically generated oligonucleotide
gatcggatccatgggaggggccgtggt

SEQ ID NO: 25 - DNA, 26 bp, Artificial Sequence
  synthetically generated oligonucleotide
gtcagatatcgccactcagacacttg

SEQ ID NO: 26 - protein, 16 aa, Homo sapiens
CQKDTEGGPKEEESPV

SEQ ID NO: 27 - protein, 17 aa, Homo sapiens [sequence omitted]

SEQ ID NO: 28 - protein, 17 aa, Homo sapiens
CTEPEIEARPRLAAESV

SEQ ID NO: 29 - protein, 17 aa, Homo sapiens
CEPEKNGEVVHTPETSV

SEQ ID NO: 30 - protein, 9 aa, Homo sapiens
AWRHPQFGG

SEQ ID NO: 31 - DNA, 82 bp, Homo sapiens
ccggaccagaaagacacagaaggagggcccaaggaggaggaaagtccagtcgcttggagacatccacaatttggtggttaat

SEQ ID NO: 32 - DNA, 82 bp, Homo sapiens
ctagattaaccaccaaattgtggatgtctccaagcgactggactttcctcctccttgggccctccttctgtgtctttctggt

SEQ ID NO: 33 - DNA, 60 bp, Homo sapiens
gtgtaacctcagaaagagaaactaacattgcttggagacatccacaatttggtggttaat

SEQ ID NO: 34 - DNA, 67 bp, Homo sapiens
ctagattaaccaccaaattgtggatgtctccaagcaatgttagtttctctttctgaggttacactct

SEQ ID NO: 35 - DNA, 20 bp, Artificial Sequence
  primer
gccatcctgctggtgaacta

SEQ ID NO: 36 - DNA, 74 bp, Artificial Sequence
  primer
tagctagtctagattaaccaccaaattgtggatgtctccaagctacagactcggcagccagcctcggcctcgcc

SEQ ID NO: 37 - protein, 509 aa, Homo sapiens
MPPAVGGPVGYTPPDGGWGWAVVIGAFISIGFSYAFPKSITVFFKEIEGIFHATTSEVSWISSIMLAVMYGGGPISSILVNKYGSRIVMIVGGCLSGCGLIAASFCNTVQQLYVCIGVIGGLGLAFNLNPALTMIGKYFYKRRPLANGLAMAGSPVFLCTLAPLNQVFFGIFGWRGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKAAESPDQKDTEGGPKEEESPVAWRHPQFGG

SEQ ID NO: 38 - protein, 487 aa, Homo sapiens
MPPMPSAPPVHPPPDGGWGWIVVGAAFISIGFSYAFPKAVTVFFKEIQQIFHTTYSEIAWISSIMLAVMYAGGPVSSVLVNKYGSRPVVIAGGLLCCLGMVLASFSSSVVQLYLTMGFITGLGLAFNLQPALTIIGKYFYRKRPMANGLAMAGSPVFLSSLAPFNQYLFNTFGWKGSFLILGSLLLNACVAGSLMRPLGPNQTTSKSKNKTGKTEDDSSPKKIKTKKSTWEKVNKYLDFSLFKHRGFLIYLSGNVIMFLGFFAPIIFLAPYAKDQGIDEYSAAFLLSVMAFVDMFARPSVGLIANSKYIRPRIQYFFSFAIMFNGVCHLLCPLAQDYTSLVLYAVFFGLGFGSVSSVLFETLMDLVGAPRFSSAVGLVTIVECGPVLLGPPLAGKLVDLTGEYKYMYMSCGAIVVAASVWLLIGNAINYRLLAKERKEENARQKTRESEPLSKSKHSEDVNVKVSNAQSVTSERETNIAWRHPQFGG

SEQ ID NO: 39 - protein, 513 aa, Homo sapiens
MGAGGPRRGEGPPDGGWGWVVLGACFVVTGFAYGFPKAVSVFFRALMRDFDAGYSDTAWVSSIMLAMLYGTGPVSSILVTRFGCRPVMLAGGLLASAGMILASFATRLLELYLTAGVLTGLGLALNFQPSLIMLGLYFERRRPLANGLAAAGSPVFLSALSPLGQQLLERFGWRGGFLLLGGLLLHCCACGAVMRPPPGPGPRPRRDSAGDRAGDAPGEAEADGAGLQLREASPRVRPRRRLLDLAVCTDRAFAVYAVTKFLMALGLFVPAILLVNYAKDAGVPDTDAAFLLSIVGFVDIVARPACGALAGLARLRPHVPYLFSLALLANGLTDLSSARARSYGALVAFCVAFGLSYGMVGALQFEVLMAAVGAPRFPSALGLVLLVEAAAVLIGPPSAGRLVDALKNYEIIFYLAGSEVALAGVFMAVATNCCLRCAKAAPSGPGTEGGASDTEDAEAEGDSEPLPVVAEEPGNLEALEVLSARGEPTEPEIEARPRLAAESVAWRHPQFGG

SEQ ID NO: 40 - protein, 500 aa, Homo sapiens
MPPMPSAPPVHPPPDGGWGWIVVGAAFISIGFSYAFPKAVTVFFKEIQQIFHTTYSEIAWISSIMLAVMYAGGPVSSVLVNKYGSRPVVIAGGLLCCLGMVLASFSSSVVQLYLTMGFITGLGLAFNLQPALTIIGKYFYRKRPMANGLAMAGSPVFLSSLAPFNQYLFNTFGWKGSFLILGGLLLNCCVAGALMRPIGPKPTKAGKDKSKASLEKAGKSGVKKDLHDANTDLIGRHPKQEKRSVFQTINQFLDLTLFTHRGFLLYLSGNVIMFFGLFAPLVFLSSYGKSQHYSSEKSAFLLSILAFVDMVARPSMGLVANTKPIRPRIQYFFAASVVANGVCHMLAPLSTTYVGFCVYAGFFGFAFGWLSSVLFETLMDLVGPQRFSSAVGLVTIVECCPVLLGPPLLGRLNDMYGDYKYTYWACGVVLIISGIYLFIGMGINYRLLAKEQKANEQKKESKEEETSIDVAGKPNEVTKAAESPDQKDTEGGPKEEESPV

SEQ ID NO: 41 - DNA, 1503 bp, Homo sapiens
atgccaccagcagttggaggtccagttggatacacccccccagatggaggctggggctgggcagtggtaattggagctttcatttccatcggcttctcttatgcatttcccaaatcaattactgtcttcttcaaagagattgaaggtatattccatgccaccaccagcgaagtgtcatggatatcctccataatgttggctgtcatgtatggtggaggtcctatcagcagtatcctggtgaataaatatggaagtcgtatagtcatgattgttggtggctgcttgtcaggctgtggcttgattgcagcttctttctgtaacaccgtacagcaactatacgtctgtattggagtcattggaggtcttgggcttgccttcaacttgaatccagctctgaccatgattggcaagtatttctacaagaggcgaccattggccaacggactggccatggcaggcagccctgtgttcctctgtactctggcccccctcaatcaggttttcttcggtatctttggatggagaggaagctttctaattcttgggggcttgctactaaactgctgtgttgctggagccctcatgcgaccaatcgggcccaagccaaccaaggcagggaaagataagtctaaagcatcccttgagaaagctggaaaatctggtgtgaaaaaagatctgcatgatgcaaatacagatcttattggaagacaccctaaacaagagaaacgatcagtcttccaaacaattaatcagttcctggacttaaccctattcacccacagaggctttttgctatacctctctggaaatgtgatcatgttttttggactctttgcacctttggtgtttcttagtagttatgggaagagtcagcattattctagtgagaagtctgccttccttctttccattctggcttttgttgacatggtagcccgaccatctatgggacttgtagccaacacaaagccaataagacctcgaattcagtatttctttgcggcttccgttgttgcaaatggagtgtgtcatatgctagcacctttatccactacctatgttggattctgtgtctatgcgggattctttggatttgccttcgggtggctcagctccgtattgtttgaaacattgatggaccttgttggaccccagaggttctccagcgctgtgggattggtgaccattgtggaatgctgtcctgtcctcctggggccaccacttttaggtcggctcaatgacatgtatggagactacaaatacacatactgggcatgtggcgtcgtcctaattatttcaggtatctatctcttcattggcatgggcatcaattatcgacttttggcaaaagaacagaaagcaaacgagcagaaaaaggaaagtaaagaggaagagaccagtatagatgttgctgggaagccaaatgaagttaccaaagcagcagaatctccggaccagaaagacacagaaggagggcccaaggaggaggaaagtccagtctga

SEQ ID NO: 42 - DNA, 1437 bp, Homo sapiens
atgccaccaatgccaagtgccccacctgtgcatccacctccagatggaggatggggttggattgtggttggagcagcttttatctccattggattttcctatgcattccccaaagctgtcaccgtattcttcaaagaaattcagcaaatattccacactacctacagtgaaatagcatggatttcatccattatgctggctgttatgtacgcaggaggtcctgtaagtagtgttttggtgaataaatacggcagccggccggtggtgatagcaggaggcttattatgctgtcttggaatggtgttggcctcctttagtagcagcgtggtacagctgtacctcactatgggattcattacaggtttaggtttagccttcaacctgcaacccgccttaaccataattggcaaatacttctataggaagcgacccatggcaaatggattggccatggcaggaagtcctgttttcttaagttcattggctcctttcaatcagtacctttttaatacttttggctggaaaggaagcttcctgattttgggaagtctacttttgaatgcctgtgtggctggttccctcatgagaccccttggacccaatcaaaccacttctaagtctaaaaataagactggcaaaacagaagatgattcaagcccaaagaaaatcaaaacgaagaaatcaacttgggaaaaagttaataagtatttagatttctccctttttaagcatagaggatttctgatatatctgtctggaaatgtcattatgttcctaggtttttttgcccccattatattcttggctccatatgctaaagaccaaggaattgatgagtactcggcagcttttctgctatctgttatggctttcgttgatatgtttgctaggccttctgtaggattaattgcaaactccaaatatattcgacctcgaattcagtacttcttcagttttgcaatcatgttcaatggagtgtgtcacctcttgtgcccactggcacaggactacacaagcctggtattatatgctgtattttttggccttggatttgggagtgttagcagtgttctctttgaaactctcatggacctcgtgggtgcaccaagattttccagtgccgtcggacttgtcacaattgtggagtgtggcccagttcttcttggccctcctcttgcaggtaaattggtggatttaactggagaatataaatacatgtacatgtcctgtggggctattgtggtagcagcaagcgtgtggctgctcattggcaatgctatcaactatagattgcttgcaaaggaaaggaaggaggaaaatgcaaggcagaagaccagagaatctgaacccttgagcaaatctaaacattcggaagatgttaacgtcaaagtttcaaatgcacagagtgtaacctcagaaagagaaactaacatttaa

SEQ ID NO: 43 - DNA, 1515 bp, Homo sapiens
atgggcgctggcggcccccggcggggcgagggccccccagacggcggctggggctgggtggtgctgggcgcctgctttgtggtcaccggcttcgcctacggcttccccaaagccgtgagcgtcttcttccgcgcgctcatgcgcgacttcgatgccggctacagcgacacggcctgggtgtcctccatcatgctagccatgctctacggcacgggccccgtgtccagcatcctcgtgacccgctttggctgtcgcccggtgatgctggcgggtgggctgctggcttccgcgggcatgatcctagcttcctttgccacgcgcctcctggagctctacctgaccgctggggtgctcacaggcctgggcctggccctcaacttccagccgtcgctcatcatgctggggctgtacttcgagcggcggcggcctctggccaacgggctggcggcggcgggcagccccgtgttcctgtccgcgctgtcgccgctcggccagcagctgctggagcgcttcggctggcgcggcggcttcctgctgctcggcgggctcctgctgcactgctgcgcctgcggggctgtcatgaggccgccgcccgggccgggcccgcgaccgcgcagggacagcgccggcgaccgcgccggggacgctccgggcgaggcggaggctgacggtgcggggctgcagctgcgcgaggcatcccccagggtccggccccgccggcgcctgctggacttggcagtgtgcaccgaccgcgccttcgccgtgtacgccgtcaccaagttcctgatggcgctcgggctcttcgtccccgccatcctactggtgaactacgccaaggacgcgggcgtgcccgacaccgacgccgccttcctgctgtccatcgtgggcttcgtggacatcgtggcgcgcccggcgtgcggcgccctggcgggcctggcgcgtctgcggccgcacgtcccgtatctgttcagcctggccctgctggccaatgggctcacagacctgagcagcgcacgcgcgcgctcctacggcgccctcgtcgccttctgcgtcgccttcggcctctcctacggcatggtgggcgcgctgcagttcgaggtgctcatggcggctgtgggcgcgccccgcttccccagtgcgctgggcctggtgttgctcgtggaggccgcggctgtgctcatcggaccgccctctgccggccgcctggtggatgcgttgaagaactatgagatcatcttctacctggccggctctgaggtggccctggctggggtcttcatggctgtcgccaccaactgctgcctgcgttgtgctaaagctgccccgtcaggcccaggcactgagggcggagccagtgacactgaggacgctgaggctgaaggggactctgagcccctgcctgttgttgcagaggaacccggcaacctggaggccctggaggtgctcagcgcccggggcgagcccacagaaccagaaatagaggcgaggccgaggctggctgccgagtctgtataa

SEQ ID NO: 44 - DNA, 1398 bp, Homo sapiens
atgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctga

(19) United States
(12) Patent Application Publication
(10) Pub. No.: US 2004/0072746 A1
(43) Pub. Date: Apr. 15, 2004

(54) Inhibitors of monocarboxylate transport

(76) Inventors: Michael Sullivan, Loughborough (GB); Clare Margaret Murray, Loughborough (GB); Raymond Hutchinson, Loughborough (GB); David Keith Donald, Loughborough (GB); Clive Geoffrey Jackson, Loughborough (GB); Andrew Paul Jackson, Loughborough (GB); John Raymond Bantick, Loughborough (GB); Ian David Cook, Waltham, MA (US)

Correspondence Address: Janis K. Fraser, Esq., Fish & Richardson P.C., 225 Franklin Street, Boston, MA, 02110-2804, US

(21) Appl. No.: 10/272,196

(22) Filed: Oct. 16, 2002

Related U.S. Application Data
(60) Provisional application No. 60/329,318, filed on Oct. 16, 2001.

(51) Int. Cl. G01N 33/574; A61K 38/17

(52) U.S. Cl. 514/12; 435/7.23

(57) ABSTRACT

Methods of screening compounds for their ability to inhibit monocarboxylate transport, and methods of making and using such inhibitors to treat disorders associated with cellular proliferation, e.g., immune-mediated disorders and cancer.

This patent application claims the benefit of U.S. Provisional Application Serial No. 60/329,318, filed 16 Oct. 2001, the specification of which is hereby incorporated by reference.

This invention relates, inter alia, to methods of screening compounds for their ability to reduce monocarboxylate transport, and methods of using such compounds to treat certain cancers and immune-mediated disorders.

### BACKGROUND OF THE INVENTION

The immune system has evolved to detect the presence of foreign organisms such as bacteria, viruses and other pathogens, and to mount protective immune responses to eliminate them. Under certain circumstances, the induction of an immune response against foreign organisms or tissues proves more harmful to the host than ignoring them. For example, asthma and allergies to food and extrinsic antigens such as pollen are believed to reflect inappropriate hypersensitivity responses to otherwise harmless substances. In addition, transplant patients often exhibit strong immune responses against transplanted tissues. These are detrimental to the survival of the transplanted organ and must be limited by administration of potent immunosuppressive drugs.

Under normal circumstances the immune system does not produce immune responses to self-tissues and self-antigens. However, under some conditions immune responses are mounted against self-tissues in such an aggressive manner that they lead to destructive autoimmune diseases: for example, rheumatoid arthritis, multiple sclerosis and type I diabetes. In these situations it would be desirable to reset the immune system so that responses to self-antigens are silenced but without affecting protective host defence mechanisms directed against exogenous antigens.

Most immune responses are initiated and controlled by helper T lymphocytes, which respond to antigenic peptide fragments presented in association with MHC Class II molecules, and cytotoxic T lymphocytes, which respond to peptides presented in association with MHC Class I molecules on specialised antigen presenting cells such as dendritic cells.

Full T cell activation requires two distinct signals from the antigen presenting cell. Signal 1 is antigen specific and is provided by the interaction of the T cell receptor (TCR) with the MHC-peptide complex displayed by the antigen-presenting cell. Signal 2 is antigen independent and involves the interaction of the co-stimulatory T cell molecule, CD28, with its ligand, B7, on antigen presenting cells. These cell-surface interactions trigger downstream biochemical signalling pathways that ultimately result in IL2 transcription and T-cell activation.

As a means of treating these immune-mediated disorders, research programs to date have typically concentrated on identifying compounds capable of blocking IL2 transcription. The promoter region of the IL2 gene includes a binding site for the Nuclear Factor of Activated T-cells (NFAT) transcription factor complex. This complex is composed of nuclear components fos and jun and a cytoplasmic component NFAT_{c} that translocates to the nucleus after dephosphorylation by the phosphatase calcineurin. The immunosuppressive macrolides cyclosporin A (CsA) and FK506 block transcription of the IL2 gene in T-lymphocytes by preventing formation of the NFAT complex (Crabtree, Cell 96:611-614, 1999). Complexes formed by the binding of CsA and FK506 to their respective immunophilins, cyclophilin and FKBP12, inhibit calcineurin activity and thus block NFAT_{c} translocation. Although CsA and FK506 are potent immunosuppressive drugs used clinically for the prevention of graft rejection, their long-term utility for the treatment of auto-immune disease is limited by their side effect profile, including nephrotoxicity. These adverse reactions appear to be related to inhibition of calcineurin activity, as the enzyme is expressed widely across mammalian tissues and has multiple functions.

A number of additional immunosuppressive therapies have now been developed (Dumont, Opin. Ther. Patents 11:377-404, 2001). These include rapamycin, which disrupts the cytokine (e.g., IL2)-driven proliferation of T-cells, by interfering with the function of TOR (Target Of Rapamycin), a kinase involved in the cytokine signalling pathway (Dumont and Su, Life Sci. 58:373-395, 1996). However, rapamycin has been shown to cause significant side effects, including thrombocytopenia and hyperlipidemia (Hong and Kahan, Semin. Nephrol. 20 (2):108-125, 2000). Antimetabolite approaches are also of utility for immunosuppression, as T-lymphocytes have been shown to be dependent on de novo synthesis of ribonucleotides (Fairbanks et al., J. Biol. Chem. 270(50):29682-29689, 1995). Mycophenolate mofetil (MMF), which inhibits inosine monophosphate dehydrogenase (IMPDH), the regulatory enzyme of guanine nucleotide biosynthesis (Allison and Eugui, Immunopharmacology 47:85-118, 2000), is effective in reducing T-cell proliferation and has been used for the treatment of graft rejection. However, MMF is rapidly glucoronidated in vivo and is associated with gastrointestinal toxicity (Dumont, Curr. Opin. Invest. Drugs. 2 (3):357-363, 2001).

Screening programmes investigating NFAT-mediated transcription directly have been used to identify small molecule inhibitors of IL2 production without the side effect profile of calcineurin inhibitors. Michne et al. discovered a class of quinazolinedione compounds that were identified by inhibition of NFAT-mediated gene transciption in a Jurkat human leukemic T-cell line (Michne et al., J. Med. Chem. 38:2557-2569, 1995). An example of the quinazolinedione class of compounds, WIN 61058, that inhibited NFAT-mediated transcription with a potency of 2 μM was shown to block IL2 production in Jurkat T-cells and to inhibit a human Mixed Lymphocyte Reaction (MLR) (Baine et al., J. Immunol. 154:3667-3677, 1995). A chemical programme based on WIN 61058 resulted in the identification of pyrrolopyrimidinedione inhibitors of NFAT-mediated transcription with potencies as high as 2 nM (Michne et al., supra). We have confirmed that examples of these pyrrolopyrimidinediones inhibit NFAT-mediated gene transcription in Jurkat T-cells, but found that early IL2 production in response to mitogenic stimulation of peripheral blood mononuclear cells (PBMC) is not significantly inhibited. The applicant's own chemical programme has exemplified pyrimidinedione inhibitors that potently inhibit the human MLR in the absence of inhibition of IL2 transcription. Contrary to the mechanism proposed by Michne et al. (supra), the inventors have, for the first time, identified the mechanism of action of these pyrimidinedione compounds as being blockade of monocarboxylate transport through the monocarboxylate transporter MCT1. This pioneering invention explains a totally new mechanism of action not foreshadowed in any way by the art in this highly competitive field, opening up new methods of treatment by inhibiting this mechanism of action and also new targets for identifying immunosuppressive agents.

WO 98/46606 (AstraZeneca), incorporated herein by reference, discloses a family of pyrazolo[3,4-d]pyrimidinedione compounds; WO 98/54190 (AstraZeneca), incorporated herein by reference, discloses a family of thieno[2,3-d]pyrimidinedione compounds; WO 98/28301(AstraZeneca), incorporated herein by reference, discloses a family of 5-substituted pyrrolo[3,4-d]pyrimidine-2,4-dione compounds; WO 99/29695 (AstraZeneca), incorporated herein by reference, discloses certain pyrrolo-, thieno-, furano-and pyrazolo-[3,4-d]pyridazinone compounds; WO 00/12514 (AstraZeneca), WO 01/83489 (AstraZeneca), PCT/GB02/03399 (AstraZeneca), PCT/GB02/03250(AstraZeneca) and GB-A-2363377 (AstraZeneca), each incorporated herein by reference, each disclose certain thieno[2,3-d]pyrimidinedione compounds. Each of these compounds, which exhibit pharmacological activity, in particular immunosuppressive activity, is disclosed for the first time herein to effect this via inhibition of monocarboxylate transport.

MCT1 is a member of a family of monocarboxylate transporters that mediate the influx and efflux of monocarboxylates, such as lactate and pyruvate, across cell membranes. The MCT proteins transport monocarboxylates by a facilitative diffusion mechanism that requires the co-transport of protons (Poole and Halestrap, Am. J.Physiol. 264:C761-C782, 1993; Halestrap and Price, Biochem. J 343:281-299, 1999). Nonselective small molecule inhibitors of the transporter have been identified, including 4,4′-di-isothiocyanatostilbene-2,2′-disulphonate (DTDS), α-cyano-4-hydroxycinnamate (CHC) and phloretin. These are non-selective inhibitors with potencies in the μM range (IC50 values at rat MCT1: DIDS=50 μM; CHC=27 μM; phloretin=1 μM)(Poole and Halestrap, supra). The MCT1 protein, a 55 kDa protein, has been enriched from rat red blood cells (Poole et al., Biochem. J 320:817-824, 1996). Hydrophobicity analysis and studies on the membrane topology of rat MCT1 have suggested a structure with 12 transmembrane segments and intracellular N- and C-terminal regions (Poole et al., supra).

The nomenclature for the MCT family is taken from Price et al., Biochem. J 329 (2):321-328, 1998; and Halestrap and Price, Biochem. J 343:281-299, 1999.

Roth et al. showed that addition of exogenous lactate to T-cells resulted in inhibition of DNA synthesis and proliferation (Roth et al., Cell. Immunol. 136:95-104, 1991) but that IL2 production was augmented (Droge et al., Cell. Immunol. 108:405-416, 1987). Studies on the metabolism of activated T-lymphocytes (thymocytes) have demonstrated that the cells derive 86% of their energy supply by aerobic glycolysis, i.e., glycolytic breakdown of glucose to lactate (Brand and Hermfisse, FASEB J 11:388-39, 1997; Guppy et al., Eur. J Biochem. 212:95-99, 1993). The monocarboxylate transporter MCT4 has been characterised as a transporter with low affinity for lactate and pyruvate (Dimmer et al., Biochem. J 350:219-227, 2000). Therefore, it has been suggested that MCT4 is adapted to the release of lactate from glycolytically-active cells, whereas the high affinity transporter MCT1 transports lactate required for energy production into cells (Manning Fox et al., J.Physiol. 529:285-293, 2000).

Zhao et al. (Diabetes 50:361-366, 2001) propose that, in some forms of Type II diabetes, MCT overexpression in the pancreatic islet cells could contribute to aberrant secretion of insulin, and, therefore postulate that inhibitors of islet cell lactate transport, or of MCT1 gene expression, could provide a therapeutic target for this disease.

Froberg et al. (Neuroreport 12(4):761-765, 2001) reported increased MCT1 expression in high grade glial neoplasms and speculated that it may provide a potential therapeutic target for treatment of some CNS neoplasms.

The monocarboxalate transporters MCT1 to MCT4 are known to transport monocarboxylate (Halestrap and Price, Biochem. J 343:281-299, 1999).

### SUMMARY OF THE INVENTION

It has now been discovered that compounds capable of blocking cellular monocarboxylate transport can inhibit cellular proliferation, and are therefore useful for treatment of various disorders associated with unwanted cellular proliferation. Accordingly, in a first aspect of the invention there is provided a method for identifying compound(s) that may have therapeutic potential, the method comprising determining whether a test compound decreases monocarboxylate transport activity, and, if the compound decreases such activity, identifying the compound as having therapeutic potential. The term “decreasing monocarboxylate transport activity” is intended to cover decreasing such activity by any means that specifically affects monocaraboxylate transport, e.g., by inhibiting the activity of an existing monocarboxylate transporter protein molecule or by reducing the amount of cellular monocarboxylate transporter protein present in a cell. Thus, the determining step might involve, for example, determining whether the test compound directly inhibits the activity of a monocarboxylate transport protein, or determining whether the compound reduces the level of expression or the total amount of a monocarboxylate transport protein in a cell. Particularly where inhibition of the protein's activity is the focus of the screening method, the protein would preferably be in a cell, a cell ghost, a cell membrane fraction, or a lipid vesicle.

Methods of carrying out the determining step can include the sub-steps of (i) providing a cell expressing a monocarboxylate transporter protein; (ii) contacting the cell with the test compound; and (iii) determining whether the test compound affects one or more of the following: monocarboxylate accumulation within the cell, monocarboxylate efflux from the cell, H+ efflux from the cell, or H+ accumulation within the cell, as an indication that the test compound inhibits the protein's monocarboxylate transport activity. The determining step optionally employs an assay selected from the group consisting of rapid filtration of equilibrium binding mixtures, radioimmunoassays (RIA), fluorescence resonance energy transfer assays (FRET), scintillation proximity assay (SPA), measurement of intracellular pH, and the use of labelled substrates to measure transport.

In this and each of the other screening methods of the invention, the preferred target transport protein is MCT1, 2, 3, or 4, preferably of a warm-blooded animal, and more preferably of a mammal such as a human, mouse, rat, guinea pig, hamster, rabbit, dog, cat, cow, horse, goat, sheep, pig, or non-human primate. The test compound can be any type of compound, including in particular small molecules as well as proteins such as antibodies or antibody fragments.

In preferred embodiment of each of the screening methods described herein, the method is useful in identifying agents(s) that may have potential in treating a disorder associated with unwanted cellular proliferation, such as an immune-mediated disorder (e.g., transplant rejection and inflammation), cancer (e.g., non-central nervous system (CNS) or non-glial cell cancers), or blood vessel blockage, as in restenosis. Accordingly, any of the screening methods can include an additional step of further testing a compound that was identified in the screen in a cellular proliferation assay. Such an assay could test, for example, whether the compound inhibitis proliferation of cells such as activated T lymphocytes, cancer cells in vitro, or cancer cells in vivo. Alternatively, the compound could be further tested in an in vivo or in vitro model of inflammation, autoimmune disease, or transplant rejection.

In another method of the invention, there is provided a method for identifying a compound having therapeutic potential, the method including the steps of (a) determining whether a test compound binds to a monocarboxylate transport protein; and (b) if the compound binds to the protein, identifying the compound as having therapeutic potential. The determining step can include ascertaining the binding affinity of the compound for the protein, optionally in accordance with the techniques described below. Alternatively or in addition, the determining step can include a competitive binding assay, using as competitive reagent a labelled second compound that specifically binds to the protein. In a preferred embodiment, the determining step comprises providing a cell expressing the protein, or a cell membrane preparation derived from the cell, and contacting the test compound with the cell or the preparation. The cell may naturally express the protein, or the protein may be a recombinant protein expressed by a cell that is transfected with a nucleic acid encoding the protein. In the latter case, the cell prior to transfection would preferably express little or no monocarboxylate transport protein of the type being studied.

According to a further aspect of the invention, there is provided a method for determining whether a compound not known to be capable of specifically binding to a monocarboxylate transporter can specifically bind to a monocarboxylate transporter. This method comprises contacting a monocarboxylate transporter protein with the compound under conditions suitable for binding, and detecting specific binding of the compound to the transporter. Such a method is particularly applicable for identifying potentially useful therapeutic compounds. In one embodiment the transporter is presented within a natural or synthetic membrane. For example, the transporter could be presented within lipid vesicles, as described by Lynch and McGiven (Biochem. J 244:503-508, 1987).

According to a further aspect of the invention, there is provided an assay for identifying compounds that inhibit monocarboxylate transport in a cell, the assay comprising:

(a) contacting a cell or cell lysate comprising a monocarboxylate transport polypeptide (or a DNA or RNA encoding the polypeptide) with a test compound; and

(b) detecting one or more of the following characteristics:

(i) the ability of the test compound to inhibit the ability of the monocarboxylate transport polypeptide to transport monocarboxylate,

(ii) the ability of the test compound to bind to the monocarboxylate transport polypeptide, and

(iii) the ability of the test compound to block expression of the monocarboxylate transport polypeptide.

According to a further aspect of the invention there is provided a method for identifying whether or not a compound may have potential in treating an immune-mediated disorder or cancer (particularly a cancer other than a CNS or glial cell cancer), which comprises contacting cells expressing a monocarboxylate transporter, or cell membrane preparations thereof, with a compound not known to be capable of inhibiting monocarboxylate transport, under conditions suitable for binding, and determining monocarboxylate transport activity, wherein the ability of the compound to inhibit monocarboxylate transport identifies that compound as having potential in treating an immune-mediated disorder or cancer.

According to a further aspect of the invention there is provided a method for determining whether a compound not known to be capable of blocking monocarboxylate transport can block monocarboxylate transport, which comprises contacting cells expressing a monocarboxylate transporter, or cell membrane preparations thereof, with the compound under conditions suitable for binding, and determining monocarboxylate transport activity. This method can be employed, for example, to determine the suitability of a compound for assessment as a potential therapeutic agent. Potential test therapeutic agents would possess IC50 values of at least 10 μM, preferably at least 1 μM, for inhibition of monocarboxylate transport (IC50 being the concentration of compound resulting in 50% inhibition of the response). In one embodiment the monocarboxylate transporter is expressed from nucleic acid exogenously introduced into a cell. In another embodiment monocarboxylate transport is blocked as a result of the compound's specifically binding to the monocarboxylate transporter. In another embodiment monocarboxylate transport is blocked as a result of the compound's impeding expression of the monocarboxylate transporter. In a further embodiment the method includes a step of determining whether or not the compound is capable of specifically blocking monocarboxylate transport.

A compound is typically identified as an MCT inhibitor if it exhibits an inhibition constant, Ki, of less than or equal to 10 μM. The inhibition constant, Ki, is the concentration of competing ligand that would occupy 50% of the binding sites if no radioligand were present in the competitive binding assay. The Ki is calculated from the IC50 using the Cheng-Prusoff equation. IC50 values are determined as the concentration of inhibitor that would displace 50% of radioligand A or C (described in Example 1 herein) as measured in filter binding assays and/or the scintillation proximity assay(s) described herein.

According to a further aspect of the invention there is provided a method for identifying a compound that may have potential in treating an immune-mediated disorder or cancer, comprising determining whether the compound is capable of inhibiting monocarboxylate transport activity of a cell.

Thus, in a further aspect of the invention there is provided use of a human monocarboxylate transporter, or a cell or cell membrane preparation comprising a monocarboxylate transporter protein, preferably one selected from the group consisting of MCT1 through MCT4, in the in vitro screening of compounds for their ability to treat an immune-mediated disorder or cancer, particularly non-CNS or non-glial cell cancer.

The term “monocarboxylate transport activity,” as used herein, refers to the ability of the transporter protein to facilitate transport of monocarboxylate molecules, such as lactate and pyruvate, across a cell membrane. Such activity can be determined using various techniques known to the person skilled in the art, some of which are specifically described herein in the context of detecting inhibition of such activity. According to a further aspect of the invention, there is provided a compound, or a pharmaceutically acceptable salt thereof, identified by any of the screening methods of the invention. In one embodiment, the compound will be capable of specifically inhibiting monocarboxylate transport; preferably, the compound would be at least ten times as active (and preferably at least 50 or 100 times as active) against one of the four types of MCT proteins (MCT1, 2, 3, and 4) as against any other of the four. In another preferred embodiment, the compound does not fall within any of Formulae I-IX as defined below. In addition, the compound preferably is not 4,4′-di-isothiocyanatostilbene-2,2′-disulphonate (DIDS), α-cyano-4-hydroxycinnamate (CHC), or phloretin. In another embodiment, the compound is not a quinazolinedione, pyrimidinedione, or pyridazinone compound. The compound is preferably not one specifically disclosed, by chemical name or by generic or specific formula, in any of the prior art referenced herein.

According to a further aspect of the invention, there is provided a method of producing a pharmaceutical composition, which method comprises determining whether or not a compound is an MCT inhibitor using any of the screening methods of the invention and furthermore mixing the compound identified therein, or a pharmaceutically active derivative thereof, with a pharmaceutically acceptable carrier. Alternatively, the method can comprise providing a compound that was identified as an MCT inhibitor using a screening assay of the invention, and mixing the compound with a pharmaceutically acceptable carrier or diluent. In another aspect, the process includes the steps of carrying out one of the screening methods disclosed herein to identify a compound with therapeutic potential, and manufacturing a therapeutic composition comprising the compound in accordance with practices that ensure the sterility of the composition (e.g., Good Manufacturing Practices espoused by regulatory agencies). The resulting composition can then be labelled for use in a method of treating a specified cell proliferative disorder, such as an immune-mediated condition or cancer. Generally, such a label would describe the disorder and how the composition should be administered to a patient in need thereof, including dosage.

In the context of this aspect of the invention, a derivative of a compound identified in a screen of the invention is a compound that has been designed, synthesised and tested for MCT inhibitor activity based on the structure of the parent compound initially identified in the screen. Such a derivative compound is generally identified using conventional structure activity relationship (SAR) studies. Furthermore, such derivative compounds will generally share significant structural features with the parent compound, but with one or more structural moieties altered. A derivative compound is likely to be one whose structure has been optimised to make the compound more suitable for therapeutic treatments, such as by removal of groups known to be associated with toxic effects; being more bioavailable; having a longer half-life in vivo, etc.

According to a further aspect of the invention there is provided a method of producing a pharmaceutical composition that comprises determining whether or not a compound is an MCT inhibitor using any of the screening methods of the invention; preparing derivative compounds of this “parent” compound; testing these derivative compounds in one of the screening methods of the invention to identify a more active compound; and mixing said more active compound identified therein with a pharmaceutically acceptable carrier.

The components of any of the screening methods of the invention can be combined in a suitable kit of parts format. Thus, according to a further aspect of the invention there is provided a kit for use in a method for screening compounds for binding to or inhibition of a MCT, the kit comprising:

(i) a vessel containing a cell capable of expressing a monocarboxylate transporter protein or a cell membrane preparation containing a monocarboxylate transport protein; and

(ii) a vessel containing a labelled compound that specifically binds to the monocarboxylate transporter protein.

The kit optionally includes instructions for use in a screening assay of the invention, and optionally describes the utility of the assay in identifying compounds that are useful for treating cancer and/or immune-mediated diseases.

The invention includes a method of treating an animal (including a human) subject in need of treatment for a disease or condition characterized by T-cell activation or cellular proliferation (e.g., an immune-mediated disorder or cancer), the method comprising administering to the subject a compound that inhibits cellular monocarboxylate transporter activity. In this and each of the other methods of treatment described herein, the compound is preferably not a compound of formulae I to IX. Alternatively, the preferred compound can be characterized as being other than a compound disclosed in any of International Publication Nos. WO 98/46606, WO 98/54190, WO 98/28301, WO 99/29695, WO 00/12514, and WO 01/83489; International Application numbers PCT/GB02/03399 and PCT/GB02/03250; and UK Patent application number GB-A-2363377. Furthermore, the compound is preferably not one disclosed in Michne et al., J. Med. Chem. 38:2557-2569 (1995); or Baine et al., J. Immunol. 154:3667-3677 (1995). It also is preferably not a quinazolinedione compound.

In another aspect, the method of treating a subject in need of treatment for an immune-mediated disorder or cancer comprises identifying a subject as being in need of such treatment and administering to the subject a compound that inhibits or otherwise decreases the activity of a monocarboxylate transporter other than MCT1 or MCT2. The transporter can be, for example, MCT3 or MCT4.

Also within the invention is a method of treating a patient suffering from or likely to suffer from an immune-mediated disorder or cancer, the method comprising (i) identifying a compound as being an inhibitor of monocarboxylate transport in a cell, and (ii) administering to the patient an effective amount of the compound. In this as well as the other treatment methods disclosed herein, the compound can be a broad spectrum inhibitor capable of potently inhibiting at least two monocarboxylate transport proteins. In another embodiment, the compound is at least ten times as active against one of MCT1, 2, 3, and 4, as against any other of the four.

According to another aspect of the invention there is provided a method of treating an immune-mediated disorder or cancer by a method comprising administration, to a human subject in need of treatment, of a compound that inhibits or otherwise decreases the activity of at least one monocarboxylate transporter selected from the group consisting of MCT3 and MCT4.

According to another aspect of the invention there is provided a method of treating an immune-mediated disorder or cancer, comprising administering an effective amount of a pharmaceutical composition comprising an MCT inhibitor identifiable or identified by a screening assay method of the invention to a subject in need thereof. In one embodiment, the MCT inhibitor is a selective inhibitor.

Also within the invention is a method of inhibiting T cell or B cell proliferation in a human, the method comprising indentifying a human in need of such inhibition, and administering to the human a compound capable of specifically inhibiting monocarboxylate transport within a T cell or B cell.

In another method for treating a patient suffering from an immune-mediated disorder or cancer, the method comprises administering to the patient an effective amount of a compound that specifically reduces expression of an MCT, the compound being selected from the group consisting of an anti-sense molecule, a ribozyme molecule, an RNAi molecule, and a triple helix forming molecule.

According to another aspect of the invention there is provided a method of treating an immune-mediated disorder or cancer, the method comprising administering an effective amount of a pharmaceutical composition comprising an MCT inhibitor identifiable or identified by

(i) contacting a monocarboxylate transporter protein with a test compound under conditions suitable for binding; and

(ii) detecting specific binding of the compound to the transporter protein; to a subject in need thereof

According to another aspect of the invention there is provided a method of treating an immune-mediated disorder or cancer, comprising

(i) contacting a monocarboxylate transporter protein with a test compound under conditions suitable for binding;

(ii) detecting specific binding of the compound to the transporter protein;

(iii) preparing a pharmaceutical composition comprising the compound; and,

(iv) administering an effective amount of the pharmaceutical composition to a subject in need thereof.

According to another aspect of the invention there is provided the use of an MCT inhibitor compound that decreases the activity of a monocarboxylate transporter, other than MCT1 and MCT2, in the treatment of an immune-mediated disorder or cancer. The compound preferably does not significantly inhibit MCT1 or MCT2.

According to another aspect of the invention there is provided the use of an MCT inhibitor compound that decreases the activity of a monocarboxylate transporter selected from the group consisting of MCT3 and MCT4, in the treatment of an immune-mediated disorder or cancer. The compound preferably does not significantly inhibit MCT1 or MCT2.

According to another aspect of the invention there is provided the use of a compound that inhibits or otherwise decreases the activity of a monocarboxylate transporter, other than a compound of Formulae I to IX or as disclosed in Michne et al., supra, or Baine et al., supra., in the manufacture of a medicament for the treatment of an immune-mediated disorder or cancer.

According to another aspect of the invention there is provided the use of an MCT inhibitor compound that decreases the activity of a monocarboxylate transporter other than MCT1 and MCT2, in the manufacture of a medicament for the treatment of an immune-mediated disorder or cancer.

According to another aspect of the invention there is provided the use of an MCT inhibitor compound that decreases the activity of a monocarboxylate transporter selected from the group consisting of MCT3 and MCT4, in the manufacture of a medicament for the treatment of an immune-mediated disorder or cancer.

According to another aspect of the invention there is provided a method of treating an immune-mediated disorder or cancer, comprising (i) in vitro testing a compound for the ability to inhibit lactate transport in a cell, and (ii) administering, to a human patient in need of treatment, an effective amount of a compound which has been identified from step (i) as a compound capable of blocking lactate transport.

According to another aspect of the invention there is provided a method of treating an immune-mediated disorder or cancer, comprising (i) testing a compound for its ability to inhibit monocarboxylate transport in a cell, and (ii) administering to a human patient suffering from or likely to suffer from an immune-mediated disorder or cancer, of an effective amount of a compound which has been identified from step (i) as a compound capable of inhibiting monocarboxylate transport.

According to another aspect of the invention there is provided a method of treating a patient suffering from an immune-mediated disorder or cancer, comprising administering to a human patient suffering from or likely to suffer from such a disease or condition of an effective amount of a compound which has been shown (or is known) to be capable of blocking or inhibiting cellular monocarboxylate transport.

### DETAILED DESCRIPTION

Potential therapeutic agents that may be tested in the screening methods described herein include simple organic molecules, commonly known as “small molecules”, for example those having a molecular weight of less than 2000 Daltons. Other potential therapeutics include peptides and antibodies. The methods of the invention may be used to screen, for example, chemical compound libraries or peptide libraries, including synthetic peptide libraries and peptide phage libraries, particularly antibody display (such as scFV or Fab) phage libraries. Other suitable compound molecules include antibodies, nucleotide sequences, and any other molecules, including nucleic acid or polypeptide mimetics, that bind to an MCT. Preferably the compound is a small molecule chemical compound. The terms compound and agent are used interchangeably herein.

The screening methods of the invention will prove useful in determining whether or not test compounds (chemical or biological) may be suitable for use, inter alia, in the treatment, including prophylactic treatment, of cancers; autoimmune, inflammatory, proliferative and hyperproliferative diseases; and other immune-mediated diseases including rejection of transplanted organs or tissues. Examples of immune-mediated disorders and cancers include:

(1) (the respiratory tract) reversible obstructive airways diseases including asthma, such as bronchial, allergic, intrinsic, extrinsic and dust asthma, particularly chronic or inveterate asthma (e.g., late asthma and airways hyper-responsiveness); bronchitis; acute, allergic, atopic and chronic rhinitis, including rhinitis caseosa, hypertrophic rhinitis, rhinitis purulenta, rhinitis sicca and rhinitis medicamentosa; membranous rhinitis including croupous, fibrinous, pseudomembranous and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;

(2) (bone and joints) rheumatoid arthritis, seronegative spondyloarthropathies (including ankylosing spondylitis, psoriatic arthritis and Reiter's disease), Behcet's syndrome, Sjogren's syndrome and systemic sclerosis;

(3) (skin) psoriasis, atopic dermatitis, contact dermatitis and other eczmatous dermitides, seborrhoetic dermitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;

(4) (gastrointestinal tract) Coeliac disease, proctitis, eosinophilic gastro-enteritis, mastocytosis, Crohn's disease, ulcerative colitis, and food-related allergies that have effects remote from the gut, e.g., migraine, rhinitis and eczema;

(5) (other tissues and systemic disease) multiple sclerosis, atherosclerosis, systemic lupus erythematosus, Hashimoto's thyroiditis, myasthenia gravis, type I (but not type II) diabetes, nephrotic syndrome, eosinophilia fascitis, hyper IgE syndrome, lepromatous leprosy, Sezary syndrome and idiopathic thrombocytopenia purpura;

(6) (allograft rejection) acute and chronic allograft rejection following, for example, transplantation of kidney, heart, liver, lung, bone marrow, skin, pancreatic islet cells, cornea and stem cells; and chronic graft versus host disease.

(7) (xenograft rejection) Hyperacute, acute and chronic xenograft rejection following, for example, transplantation of kidney, heart, liver, lung, bone marrow, skin, pancreatic islet cells, cornea and stem cells; and chronic graft versus host disease.

(8) (cancer) carcinoma, including that of the bladder, breast, colon, kidney, liver, lung, ovary, pancreas, stomach, cervix, thyroid and skin; hematopoietic tumors of lymphoid lineage, including acute lymphocytic leukemia, B cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; and other tumors, including melanoma, seminoma, tetratocarcinoma, neuroblastoma and glioma.

The compounds are thus indicated for use in the treatment or prevention of rejection of transplanted organs, tissues, or cells such as kidney, heart, lung, bone marrow, skin, pancreatic islet cells, cornea and stem cells; and of autoimmune, inflammatory, proliferative, and hyperproliferative diseases, including cancer (preferably other than CNS cancers, and more particularly other than glial cell cancers), and of cutaneous manifestations of immune-mediated disorders: for example, rheumatoid arthritis, systemic lupus erythematosus, Hashimoto's thyroiditis, multiple sclerosis, myasthenia gravis, type 1 diabetes, uveitis, nephrotic syndrome, psoriasis, atopic dermatitis, contact dermatitis and further eczematous dermatitides, seborrhoeic dermatitis, Lichen planus, Pemphigus, Epidermolysis bullosa, urticaria, angioedemas, vasculitides, erythemas, cutaneous eosinophilias, Alopecia areata, eosinophilic fasciitis and atherosclerosis.

The inventors have found that compounds that are capable of binding to MCT1 and/or MCT2, and that inhibit lactate transport of activated T cells, inhibit the proliferation of activated T-cells and tumour cell lines such as the erythroleukaemia cell line K562. The first aspect of the invention is therefore a screening method to identify compounds that may be useful, inter alia, in treating conditions or diseases involving T-cell activation, such as transplant rejection and rheumatoid arthritis, or cellular proliferation, such as cancer.

In one embodiment the compound is tested for its ability to inhibit MCT activity. This, for example, may be via inhibition of the ability to transport monocarboxylates or via blockage of MCT expression. It is well known that MCT proteins from different species have a high degree of sequence similarity. For example, rat MCT1 is reported to possess 86% identity with human MCT1 (Jackson et al., Biochem Biophys Acta 1238:193-196, 1995). Thus, whilst in a preferred embodiment the MCT is of human origin, particularly from the group consisting of human MCT1, 2, 3 and 4, it is envisaged that MCTs from other species such as rat or mouse would also work in the invention. Indeed, the inventors have found that the screening method works equally well using rat MCT1 protein. Suitable monocarboxylate transporters for use in the screening assay/method of the invention include MCT1, MCT2 and MCT4.

The sequence of MCT1 is disclosed in the EMBL/GenBank/DDBJ databases (Blum H., Bauersachs S., Mewes H. W., Weil B., Wiemann S, Submitted (15 Mar. 2000) to the EMBL/GenBank/DDBJ databases) with the EMBL Accession No. AL162079. The sequence of the cDNA clone encoding human MCT1 used herein is disclosed in SEQ ID NO: 1, and is identical to the sequence disclosed by Blum et al. (supra).

With regard to MCT2, there appears to be no single definitive published sequence. Two MCT2 sequences deposited in EMBL (Accession Numbers AF049608 and AF058056) differ in three locations that lead to amino acid changes. The sequence of the cDNA clone encoding human MCT2 used herein, and disclosed in SEQ ID NO: 2, is a combination of the two. The specific amino acid differences are as follows: (i) AF049608 encodes the amino acid Asparagine at position 154, whilst AF058056 and SEQ ID NO:2 both encode a Serine at this position; (ii) AF049608 encodes the amino acid Proline at position 268, whilst AF058056 and SEQ ID NO:2 both encode a Leucine at this position; and (iii) AF058056 encodes the amino acid Serine at position 445, whilst AF049608 and SEQ ID NO:2 both encode a Threonine at this position. There are no differences between SEQ ID NO:2 and the MCT2 genomic exon and predicted transcript sequences (UCSC SOFTBERRY Database Accession No. C12001042), which confirms that the MCT2 cDNA clone depicted in SEQ ID NO:2 is native.

With regard to MCT3, compared to the predicted MCT3 amino acid sequence disclosed in Yoon et al. (Genomics. 60(3):366-370, 1990), the sequence used herein (SEQ ID NO:3) has an amino acid substitution of Tryptophan to Arginine at position 235. The published MCT3 genomic sequence (Accession No. AL031587) is consistent with the sequence of SEQ ID NO:3, encoding an Arginine at position 235.

The screening assay is not restricted to use of the full-length MCT proteins as represented in Table 1, but extends to functional variants, including mutants, deletions and chimaeric variants that maintain activity in the test assay.

TABLE 1

| Human MCT | Reference | Accession No. |
|---|---|---|
| MCT1 | Blum H., Bauersachs S., Mewes H. W., Weil B., | EMBL AL162079 |
| | Wiemann S, Submitted (15-MAR-2000) to the | |
| | EMBL/GenBank/DDBJ databases | |
| MCT2 | Journal of Biological Chemistry 273 (44), 28959- | EMBL AF049608 |
| | 28965 (1998) Lin, R. Y., Vera, J. C., Chaganti, | |
| | R. S. K. and Golde, D. W. | |
| | Human monocarboxylate transporter 2 (MCT2) is a | |
| | high affinity pyruvate transporter. | |
| | Dao L., Landschulz W. H., Landschulz K. T.; | EMBL AF058056 |
| | “Cloning of Human Monocarboxylate Transporter 2 | |
| | (hMCT2)”; Unpublished. Submitted (07-APR- | |
| | 1998) to the EMBL/GenBank/DDBJ databases. | |
| | Genomic and predicted transcript sequence for | UCSC SOFTBERRY |
| | MCT2; UCSC SOFTBERRY Database Accession | C12001042 |
| | No. C12001042 | |
| MCT3 | Genomics 60 (3), 366-370 (1999) | EMBL AF132610 |
| | Yoon, H., Donoso, L. A. and Philp, N. J. | |
| | Cloning of the human monocarboxylate transporter | |
| | MCT3 gene: localization to chromosome 22q12.3- | |
| | q13.2. | |
| | Phillimore B.; Submitted (08-DEC-1999) to the | EMBL AL031587 |
| | EMBL/GenBank/DDBJ databases | |
| MCT4 | Biochem. J 329 (Pt 2), 321-328 (1998) | EMBL U81800 |
| | Price, N. T., Jackson, V. N. and Halestrap, A. P. | |
| | Cloning and sequencing of four new mammalian | |
| | monocarboxylate transporter (MCT) homologues | |
| | confirms the existence of a transporter family with | |
| | an ancient past. | |

Each of these publications is incorporated herein by reference.

The invention is not restricted to the use of full-length native human MCTs. The use of suitable functional variants also forms part of this invention. In particular, included within the scope of the present invention is use of alleles of the disclosed MCT proteins. As used herein, an “allele” or “allelic sequence” is a naturally occurring alternative form of the molecule described herein. Alleles result from nucleic acid mutations and mRNA splice-variants, which produce polypeptides whose structure or function may or may not be altered. Any given gene may have none, one or many allelic forms. Common mutational changes that give rise to alleles are generally ascribed to natural deletions, additions or substitutions of amino acids. Each of these types of changes may occur alone, or in combination with the others, one or more times in a given sequence.

Also useful in the invention are artificially produced polypeptides that share sequence homology with, and consequently have substantially the same ligand binding ability or monocarboxylate transport activity as, the proteins coded for by the nucleotide sequence depicted in SEQ ID Nos 1 to 4. Within this category are truncated or mutated versions of the disclosed MCT proteins and chimeric polypeptides including some MCT sequence and some heterologous sequence. Such polypeptides will preferably be substantially homologous to the disclosed MCT proteins. By the term “substantially homologous,” we mean a sequence that possesses at least 70%, and in increasing order of preference at least 75%, 80%, 85%, 90%, 95%, 97% and 99% sequence identity thereto. By the term “substantially the same biological activity,” we mean having the ability to effect monocarboxylate transport. In a preferred embodiment the variant proteins will have at least 10%, and in increasing order of preference at least 20%, 30%, 40%, 50%, 60%, 70%, 80%, 90% and 95% of the monocarboxylate transportation activity of the respective wild-type protein when recombinantly expressed in a suitable heterologous expression system.

The sequence identity between two sequences can be determined by pair-wise computer alignment analysis, using the BestFit program. In practice, when searching for similar/identical sequences to the query search, from within a sequence database, it is generally necessary to perform an initial identification of similar sequences using suitable software such as Blast, Blast2, NCBI Blast2, WashU Blast2, FastA, Fasta3, PILEUP and CLUSTALW, and a scoring matrix such as Blosum 62. Such software packages endeavour to closely approximate the “gold-standard” alignment algorithm of Smith-Waterman. Thus, the preferred software/search engine programme for use in assessing similarity, i.e., how two primary polypeptide sequences line up, is Smith-Waterman. Identity refers to direct matches, similarity allows for conservative substitutions.

As used herein, the term “isolated” refers to molecules, either nucleic acid or amino acid sequences, that (1) are removed from their natural environment and purified or separated from at least one other component with which they are naturally associated, or (2) are artificially synthesised, or (3) are recombinantly produced in a milieu in which they do not naturally occur (e.g., a human MCT protein expressed in a non-human cell, or in a human cell different from a type of cell that normally expresses it); or (4) possess a sequence that differs from any known naturally occurring sequence.

Although the natural polypeptide of SEQ ID NO. 1 and a variant polypeptide may only possess for example 80% identity, they are actually likely to possess a higher degree of similarity, depending on the number of dissimilar codons that are conservative changes. Similarity between two sequences includes direct matches as well as conserved amino acid substitutes that possess similar structural or chemical properties, e.g., similar charge. Examples of conservative changes (conservative amino acid substitutes) are shown in Table 2.

Suitable conservative substitutions of amino acids are known to those of skill in this art and generally may be made without significantly altering the biological activity of the resulting polypeptide, regardless of the chosen method of synthesis. The phrase “conservative substitution” includes the use of a chemically derivatized residue in place of a non-derivatized residue, provided that the resulting polypeptide displays the desired binding activity. D-isomers as well as other known derivatives may also be substituted for the naturally occurring amino acids. See, e.g., U.S. Pat. No. 5,652,369, Amino Acid Derivatives, issued Jul. 29, 1997. Substitutions are preferably, although not exclusively, made in accordance with those set forth in TABLE 2 as follows:

TABLE 2

| Original residue | Example conservative substitution |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

The MCT coding nucleotide sequences for use in the present invention may also be engineered in order to alter a coding sequence for a variety of reasons, including but not limited to alterations that modify the cloning, processing and/or expression of the gene product. For example, mutations may be introduced using techniques that are well known in the art, e.g., site-directed mutagenesis to insert new restriction sites, to alter glycosylation patterns, to change codon preference, etc.

Because the MCT proteins are predicted to have a transmembrane domain structure, they will likely require a membrane scaffold to retain their structural and/or functional integrity. Whilst the preferred assay methods involve use of whole cells, or cell membrane preparations thereof, that contain one or more MCTs, it will be appreciated that the MCT proteins or polypeptides can be presented in alternate formats to retain their structural integrity. For example, the proteins may be reconstituted within lipid vesicles (see, for example, Lynch and McGiven, Biochem. J 244:503-508, 1987). Such alternate means of presenting the monocarboxylate transporter protein are also part of the invention. Whole cells expressing MCT or membrane preparations thereof are particularly useful. Suitable whole cells may either be natural cells or cell lines that comprise endogenous MCTs, such as Jurkat, K562, HeLa, and Chinese Hamster Ovary (CHO) cells, or transformed/transfected cells such as INS1 and SF9 cells, wherein the MCT protein has been introduced via recombinant techniques well known to the person skilled in the art.

In one embodiment, cells or cellular membrane preparations containing an MCT protein derived from cells, preferably eukaryotic, particularly mammalian, transformed, transfected or transduced with a recombinant expression construct comprising the nucleotide sequence coding for an MCT protein and sequences sufficient to direct the synthesis of the MCT protein in cultures of said transformed, transfected or transduced cells, are used to determine the binding properties of test compounds in vitro.

In one particular embodiment, the MCT protein is expressed in eukaryotic cells, especially mammalian, insect or yeast cells. Eukaryotic cells provide post-translational modifications to recombinantly expressed proteins, including folding and/or phosphorylation and/or glycosylation.

Nucleic acids coding for an MCT for use in the invention can be either isolated (e.g., from a cDNA library) or synthesised, and a variety of expression vector/host systems may be used to express MCT coding sequences. These include but are not limited to microorganisms such as bacteria transformed with plasmids, cosmids or bacteriophage; yeast transformed with expression vectors; insect cells transformed with either the baculovirus expression system or insect expression plasmids; plant cells transfected with plant virus expression systems, such as cauliflower mosaic virus; or mammalian cell systems (for example those transfected or transduced with plasmid or viral derived expression vectors, e.g., retroviral, pox, or adenoviral vectors). Selection of the most appropriate system is a matter of choice.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. The coding sequence of the polypeptide is placed under the control of an appropriate promoter, control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell; in general, signal peptides do not appear necessary for MCT proteins that end up imbedded in the cellular membrane. Preferred vectors will usually comprise at least one multiple cloning site to facilitate cloning of the gene.

Methods for the expression of MCT proteins in host cells from cloned genes is well known to those skilled in the art of molecular biology and general techniques are described in such publications as Molecular Cloning—A Laboratory Manual, Second Edition, Sambrook, Fritsch and Maniatis (Cold Spring Harbor Laboratory, 1989) and Current Protocols in Molecular Biology, Volumes1-3, Edited by F M Asubel, R Brent, R E Kingston (pub John Wiley 1998). Examples of host cells that may be transformed or transfected with nucleic acid encoding an MCT protein so as to express said MCT protein include prokaryotic cells, e.g., bacterial cells such as Escherichia coli and Bacillus subtilis; lower eukaryotic cells, e.g., yeasts such as Saccharomyces cerevisiae, Schizosaccharmoyces pombe, Pichia pastoris, Candida albicans, Aspergillus nidulans or Neurospora crassa; higher eukaryotic cells, e.g., mammalian cells such as CHO, NIH-3T3, HEK-293, Jurkat, and INS-1; insect cells such as Spodoptera frugiperda 9 and 21 cell lines; and amphibian cells such as Xenopus laevis oocytes. Performance of the invention is neither dependent on nor limited to any particular strain or type of host cell or vector; those suitable for use in the invention will be apparent to, and a matter of choice for, the person skilled in the art.

Host cells transformed or transfected with a vector containing an MCT nucleotide sequence may be cultured under conditions suitable for expression and recovery of membrane fractions containing the encoded proteins from the cell culture. Such expressed proteins will preferably, but not necessarily, be presented on the cell surface. Native cells lines used for detecting binding to MCTs or functional activity in MCTs include, e.g., K562 (human erythroleukaemia cell line) and MB231 (breast carcinoma cell line). Primary cells that naturally express MCTs may also be used for detecting binding to MCTs or functional activity in MCTs. Such primary cell types are known in the art; others can be identified by, e.g., immunoaffinity, Western blots, or Northern blots.

Membrane preparations for use in the invention can be made using standard techniques well known to the person skilled in the art, including the method disclosed in Example 10.

It will be appreciated that there are many screening methods that may be employed to determine the ability of a test compound to decrease monocarboxylate transport. Indeed, monocarboxylate transport activity can be measured directly or indirectly in a number of ways that will be apparent to the person skilled in the art. This invention incorporates each of these different ways. For example, direct binding to an MCT, such as MCT1 protein, can be determined by standard ligand binding assays. Such assays can be performed using whole cells or cell membrane preparations containing MCT proteins. Suitable alternative assays might measure monocarboxylate accumulation within the cell, monocarboxylate efflux from the cell, H+ efflux or accumulation, alterations in the glycolytic rate due to monocarboxylate feedback regulation, decreased DNA synthesis and/or cell division, and the like. Examples of suitable screening methods that may be used to identify an inhibitor of monocarboxylate transport include rapid filtration of equilibrium binding mixtures, radioimmunoassays (RIA) and fluorescence resonance energy transfer assays (FRET). A particularly useful method for identifying a compound capable of inhibiting monocarboxylate transport is a scintillation proximity assay (SPA).

SPA involves the use of fluoromicrospheres coated with acceptor molecules, such as receptors, to which a ligand will bind selectively in a reversible manner (N Bosworth & P Towers, Nature 341:167-168, 1989). The technique requires the use of a ligand labelled with an isotope that emits low energy radiation that is dissipated easily into an aqueous medium. At any point during an assay, bound labelled ligands will be in close proximity to the fluoromicrospheres, allowing the emitted energy to activate the fluor and produce light. In contrast, the vast majority of unbound labelled ligands will be too far from the fluoromicrospheres to enable the transfer of energy. Bound ligands produce light but free ligands do not, allowing the extent of ligand binding to be measured without the need to separate bound and free ligand.

The following disclosure of suitable screening methods is merely intended to be an overview, and is not intended to reflect the full state of the art. Measurement of lactate efflux/accumulation by:

1) Enzymatic measurement of lactate levels using lactate as a substrate for lactate oxidase or lactate dehydrogenase using commercially available kits such as the Sigma LO kit (735-10) or Sigma LD kit (826); or a glucose/lactate analyser (YSI 2700 analyser).

2) Transport of [¹⁴C]lactate (or another radiolabelled substrate of the monocarboxylate transporter, e.g., pyruvate, β-hydroxybutyrate, or glycolate) in an assay such as that described by Poole and Halestrap in Am. J. Physiol. 264:C761-C782 (1993).

3) Lactate-induced decrease in intracellular pH using pH sensitive dyes, e.g., 2′,7′-bis(carboxyethyl)-5(6)-carboxyfluorescein (BCECF), in an assay such as that described by Carpenter and Halestrap in Biochem. J 304:751-760 (1999).

4) Lactate-induced decrease in intracellular pH using pH-sensitive electrodes in an assay such as that described by Bröer et al. in Biochem. J 333:167-174 (1998).

5) Measurement of proton efflux/accumulation using a microphysiometer in an assay such as that described, for example, by McConnell et al., Science 257(5078):1906-1912 (1992). Each of these publications is incorporated herein by reference.

In one embodiment, the screening assay method is a competitive binding assay. Thus, according to a further aspect of the invention there is provided a competitive binding assay for compounds that may have potential in treating an immune-mediated disorder or cancer, which comprises contacting host cells expressing MCT protein, or a membrane preparation thereof, with both a first test compound and a labelled second compound known to specifically bind to said MCT protein, under conditions suitable for binding of both compounds, and detecting specific binding of the first compound to the MCT protein by measuring a decrease in the binding of the second compound to the MCT protein in the presence of the first compound, indicating that the first compound binds to the MCT protein and may thus have potential in treating an immune-mediated disorder or cancer.

As a comparative control, the assay can be conducted with only the second compound.

The first compound is preferably a small molecule compound and the second compound is preferably either a small molecule compound or an antibody. (The term “small molecule compound” is given its standard meaning in the pharmaceutical industry; it generally is meant to exclude large biological molecules such as polypeptides and nucleic acids. Typically “small molecule compounds” are at least partially chemically synthesized.) In a further embodiment, the MCT is a human MCT. In a further embodiment the MCT is selected from the group consisting of MCT1, 2, 3, and 4. Preferably, the MCT is human MCT1.

There are many conventional detectable labels, such as radioisotopes, fluorescent labels, chemiluminescent compounds, labelled binding proteins, magnetic labels, spectroscopic markers and linked enzymes that might be used to label up the second compound. Fluorescent labels are often preferred because they are less hazardous than radiolabels, they provide a strong signal with low background, and various different fluorophors capable of absorbing light at different wavelengths and/or giving off different colour signals exist to enable comparative analysis in the same analysis. For example, fluorescein gives off a green colour, rhodamine gives off a red colour, and both together give off a yellow colour. For use in the present invention, preferred labels are radioisotopes, particularly ¹⁴C, ³H and ¹²⁵I, or non-radioactive labels such as digoxigenin or biotin. The choice of label and the means of detecting such label (such as via autoradiography or fluorescence microscopy) can be made by the person skilled in the art.

In one embodiment of the invention a radioligand binding assay is performed, which comprises contacting the test compound with a cellular membrane preparation containing an MCT, preferably MCT1, and a radio-labelled ligand that is known to bind to said MCT, and measuring displacement of said ligand by the test compound.

In one embodiment, the test compounds will be specific for a particular MCT subtype. Compounds are deemed specific if they bind to one particular MCT subtype and exhibit at least 10-fold lower potency (Ki), preferably at least 25-fold lower potency, and more preferably at least 100-fold lower potency to all other subtypes.

Potential drug candidates are identified by choosing chemical compounds that bind with high affinity (IC50 of less than 10 μM) to the expressed MCT, by using, for example, ligand binding methods well known to those skilled in the art, examples of which are shown in the binding assays described herein. Drug candidates may have broad specificity acting on more than one MCT subtype; alternatively, the drug candidates will be specific for a particular MCT subtype. Compounds are deemed specific if they inhibit monocarboxylate transport by one particular MCT subtype at least ten fold, preferably at least 25 fold, and more preferably at least 100 fold more strongly than any other MCT subtype. Alternatively, compounds may be deemed specific if they bind at least ten fold, preferably at least 25 fold and more preferably at least 100 fold more strongly to one particular MCT subtype than to any other MCT subtype.

Ligands A and C (described in Example 1) are examples of suitable radioligands that can be used in the invention. Such radioligands can be made by standard techniques. These radioligands are a further aspect of the invention.

Thus, according to a further aspect of the invention there is provided a radiolabeled compound capable of binding to an MCT. In terms of a radioligand for binding to human MCT1, any of the compounds disclosed in any of WO 98/46606, WO 98/54190, WO 98/28301, WO 99/29695, WO 00/12514, WO 01/83489; PCT/GB02/03399, PCT/GB02/03250; and GB-A-2363377 could be used. In one embodiment, the radiolabelled compound is selected from the group consisting of ligands A, B and C (as described in Example 1 herein). In another aspect of the invention, there is provided the use of a radiolabelled compound capable of binding to an MCT in a screening assay to identify compounds capable of binding said MCT. In one embodiment, the MCT is MCT1, in another, the radiolabelled compound is ligand A, B or C, as described herein.

The MCT proteins or convenient fragments thereof may be used to raise antibodies. Such antibodies have a number of uses that will be evident to the molecular biologist or immunologist of ordinary skill. Such uses include, but are not limited to, use as a biotherapeutic, use as the competitive binding ligand in the screening methods of the invention, and monitoring protein expression. Enzyme linked immunosorbant assays (ELISAs) are well known in the art and would be particularly suitable for detecting the MCT polypeptide or fragments thereof. The term antibody includes both monoclonal antibodies, which are a substantially homogeneous population, and polyclonal antibodies, which are heterogeneous populations. The term also includes, inter alia, humanised and chimeric antibodies, as well as the various types of antibody constructs such as for example F(ab′)₂, Fab and single chain Fv, including bacteriophage derived antibodies.

In one embodiment, such antibodies are labelled. Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)).

Polyclonal antibodies can be readily generated from a variety of sources, for example, horses, cows, goats, sheep, dogs, chickens, rabbits, mice or rats, using procedures that are well known in the art. In general, antigen is administered to the host animal, typically through parenteral injection. Depending on the host species, various adjuvants may be used to enhance the immunological response against the injected polypeptide. Suitable adjuvants include but are not limited to Freund's (complete and incomplete), aluminium hydroxide, BCG and SAC (Bacille Calmette-Guerin and Staphylococcus aureus Cowan). Following booster immunizations, small samples of serum are collected and tested for reactivity to antigen. Examples of various assays useful for such determination include those described in Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immunosorbent assays (ELISA), dot blot assays, and sandwich assays (see, e.g., U.S. Pat. Nos. 4,376,110 and 4,486,530).

Monoclonal antibodies may be readily prepared using well-known procedures, see for example, the procedures described in U.S. Pat. Nos. RE 32,011; 4,902,614; 4,543,439 and 4,411,993; and Monoclonal Antibodies, Hybridomas: A New Dimension in Biological Analyses, Plenum Press, Kennett, McKearn, and Bechtol (eds.), (1980). By way of example, for the production of human monoclonal antibodies, hybridoma cells may be prepared by fusing spleen cells from an immunised animal, e.g., a mouse, with a tumour cell. Appropriately secreting hybridoma cells may thereafter be selected (Koehler & Milstein. Nature. 256:495-497, 1975; Cole et al. “Monoclonal antibodies and Cancer Therapy”, Alan R Liss Inc, New York N.Y. pp 77-96, 1985). Such antibodies may be of any immunoglobulin class including IgG, IgM, IgE, IgA, IgD and any subclass thereof. Rodent antibodies may be humanised using recombinant DNA technology according to techniques known in the art.

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology (1990) 3:1-9, which is incorporated herein by reference. Similarly, binding partners can be constructed using recombinant DNA techniques to incorporate the variable regions of a gene that encodes a specific binding antibody. Such a technique is described in Larrick et al., Biotechnology (1989) 7: 394.

Alternatively, chimeric antibodies, single chain antibodies (see for example, U.S. Pat. No. 4,946,778), or Fab fragments may also be developed against the polypeptides utilized in the invention (Huse et al., Science. 256:1275-1281, 1989), using techniques known in the art.

Antibodies are defined to be specifically binding if they bind the particular MCT with a Kₐ of greater than or equal to about 10⁷ M⁻¹. Affinity of binding can be determined using conventional techniques, for example those described by Scatchard et al., Ann. N.Y. Acad. Sci., (1949) 51:660.

Once isolated and purified, the antibodies may be used to detect the presence of antigen in a sample using established assay protocols, see for example “A Practical Guide to ELISA” by D. M. Kemeny, Pergamon Press, Oxford, England. Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)).

Method of Treatment

The inventors' radioligand studies indicate that the actual mechanism of action by which the compounds in International Publication Nos. WO 98/46606, WO 98/54190, WO 98/28301, WO 99/29695, WO 00/12514, and WO 01/83489; International Application numbers PCT/GB02/03399 and PCT/GB02/03250 and GB-A-2363377, each incorporated herein by reference, operate, is via binding to and inhibiting MCT1 and to a lesser extent MCT2, leading inter alia, to a buildup of lactate in the cell.

The compounds identified in these prior art patents fall within the scope of one or other of Formulae I to IX:

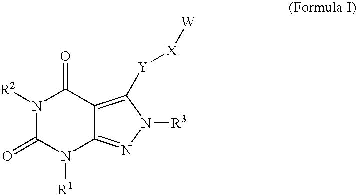

in which:

R¹ is C₁₋₆alkyl, C₃₋₆alkenyl or C₃₋₆cycloalkyl;

R² is C₁₋₄alkyl or C₃₋₆alkenyl;

R³ is 1- or 2-indanyl, 1- or 2-(1,2,3,4-tetrahydronaphthalenyl), 9-fluorenyl, acenaphthyl or CHR⁴(CH₂)ₙAr where n is 0 or 1, R⁴ is hydrogen or C₁₋₆alkyl and Ar is quinolinyl, naphthalenyl, benzodioxolinyl optionally susbstituted by one or more halogen atoms, or phenyl optionally substituted by one or more substituent groups selected from halogen, C₁₋₆alkyl, C₁₋₆alkoxy and phenylsulfonylmethyl;

W is H, CH₂OH, CO₂H, CO₂C₁₋₆alkyl, CH₂NR⁵R⁶, CONR⁵R⁶, where R⁵ and R⁶ are independently hydrogen or C₁₋₆alkyl, or together with the nitrogen atom to which they are attached form a 3- to 8-membered heterocyclic ring optionally further containing an oxygen atom or a group NR⁷ where R⁷is hydrogen or C₁₋₆alkyl, or W is pyridyl or phenyl, each of which may be optionally substituted by one or more substituent groups selected from halogen, hydroxyl, C₁₋₆alkyl and C₁₋₆alkoxy;

X is a bond or C₁₋₅alkylene;

Y is S(O)ₚ, C≡C, CH═CH, CH₂CH₂ or CH₂CH═CH; and

p is 0, 1 or 2;

or a pharmaceutically acceptable salt thereof, provided that:

X is not a bond when W is H, CH₂OH, CO₂H, CO₂C₁₋₆alkyl, CH₂NR⁵R⁶ or CONR⁵R⁶ and Y is sulfur.

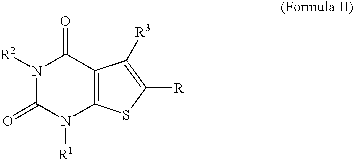

wherein:

R is —C(O)Ar¹, —C(R⁴)(R⁵)Ar¹, or Ar²;

Ar¹ is naphthyl, quinolyl, isoquinolyl, indolyl, benzofuranyl or benzothienyl, each of which can be optionally substituted by one or more substituents selected from C₁₋₄ alkyl, C₁₋₄ alkoxy, halogen or trifluoromethyl, or Ar¹ is phenyl optionally substituted by one or more substituents selected from C₁₋₄ alkyl, C₁₋₄ alkoxy, halogen, trifluoromethyl, amino, nitro, cyano, trifluoromethoxy, phenoxy, —CH₂N(R⁶)₂, —NHSO₂CF₃, C₁₋₄alkylsulphonylamino, —NHC(O)R^{6a}, CO₂R⁷ or —C(O)NR⁸R^{8a};

R⁴ represents H or C₁₋₄ alkyl;

R⁵ represents H or OH;

each R⁶ independently represents H or C₁₋₄ alkyl;

R^{6a} represents H, C₁₋₆ alkyl, aryl or arC₁₋₄alkyl, wherein the aryl group or aryl moiety in the aralkyl group is phenyl or pyridyl, each of which may be optionally substituted by one or more substituents selected from C₁₋₄ alkyl, C₁₋₄ alkoxy, C₁₋₄ alkylcarbonylamino, halogen or trifluoromethyl;

R⁷represents H or C₁₋₄ alkyl;

R⁸ and R^{8a} each independently represent H, C₁₋₄ alkyl, phenyl or pyridyl;

Ar² is acenaphthenyl, indanyl, iminodihydrobenzofuranyl or fluorenyl, each of which can be optionally substituted by one or more substituents selected from OH, C₁₋₄ alkyl, C₁₋₄ alkoxy, halogen, or trifluoromethyl;

R¹ and R² are independently H, C₁₋₆ alkyl, C₃₋₆ alkenyl, CH₂C₃₋₅ cycloalkyl or C₃₋₆ cycloalkyl;

R³represents H, X—R⁹ or X—Ar³;

X represents S(O)ₙ, C(O)NR¹⁰, C(O)O, NH(CO)NR¹⁰, NH(CO)O or SO₂NR¹⁰;

n is 0, 1 or 2;

R⁹ represents a methyl group optionally substituted by one or more substituents selected from CN, CO₂H, C₁₋₅ alkoxycarbonyl, 5-tetrazolyl, SO₂NH₂ or C(O)NR¹¹R¹², or R⁹ represents C₂₋₆ alkyl or C₃₋₆ alkenyl, each of which may be optionally substituted by one or more substituents selected from OH, CN, CO₂H, C₁₋₅ alkoxy, C₁₋₅ alkoxycarbonyl, 5-tetrazolyl, azide, phthalimido, SO₂NH₂, C(O)NR¹¹R¹², NR¹³R¹⁴, NHC(O)R¹⁵ or NHSO₂R¹⁶ where R¹¹, R¹², R¹³ and R¹⁴ each independently represent H or C₁₋₄ alkyl,

R¹⁵ represents C₁₋₄ alkyl, C₁₋₄ alkoxy, di(C₁₋₄alkyl)amino, or alkoxyalkylene containing up to 6 carbon atoms, and R¹⁶ represents C₁₋₄ alkyl or trifluoromethyl; or, additionally, in the case where X represents C(O)NR¹⁰, NH(CO)NR¹⁰ or SO₂NR¹⁰, R⁹ and R¹⁰ together with the nitrogen atom to which they are attached may form a 4- to 7-membered heterocyclic ring which may be optionally substituted by one or more OH groups;

R¹⁰ represents H, C₁₋₆ alkyl or is linked to R⁹ as defined above; and

Ar³ is phenyl, pyridyl or pyridine N-oxide, each of which may be optionally substituted by one or more substituents selected from OH, NO₂, NH₂, NHSO₂CF₃, C₁₋₄ alkoxy, bis-C₁₋₄alkanesulphonylamino, C₁₋₄alkylcarbonylamino or C₁₋₄alkoxycarbonylamino; or a pharmaceutically-acceptable salt or solvate thereof.

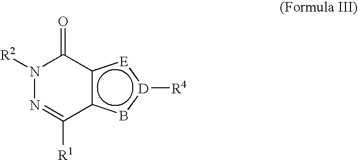

wherein B represents a group CH or a nitrogen (N), sulfur (S) or oxygen (O) atom; D represents a carbon (C) or nitrogen (N) atom; E represents a group CR³ or a nitrogen (N) atom; when D is a carbon atom, then B is a sulfur or oxygen atom and E is a group CR³, and when D is a nitrogen atom, then either B is a group CH and E is a group CR³ or a nitrogen atom, or B is a nitrogen atom and E is a group CR³; R¹ represents a group NR′R″ where R′ represent a hydrogen atom or a C₁-C₆ alkyl group, R″ represents a C₁-C₆ alkyl group, or R′ and R″ together with the nitrogen atom to which they are attached form a 3- to 7-membered saturated heterocyclic ring, or R¹ represents a C₁-C₆ alkyl, C₁-C₆ alkoxy, C₁-C₃-alkyloxyC₁-C₃-alkyl, C₃-C₆-cycloalkyloxyC₁-C₃-alkyl, C₃-C₆ alkenyl, phenyl, C₃-C₇ cycloalkyl, C₃-C₅ cycloalkylmethyl or C₃-C₇ cycloalkenyl group, each of which may be optionally substituted by one or more halogen atoms; R² represents a methyl group, or a C₂-C₆ alkyl group optionally substituted by a C₁-C₆ alkoxy group other than in the 1-position; R³ represents a hydrogen atom or a group X—R⁵ or X—Ar¹; X represents a group —O—, S(O)ₙ, SO₂N(R⁶) or C(═O)N(R⁶); n is 0, 1 or 2; R⁵ represents an optionally substituted alkyl or alkenyl group, or, additionally, in the case where X represents SO₂N(R⁶) or C(═O)N(R⁶), R⁵ and R⁶ together with the nitrogen atom to which they are attached may form an optionally substituted 3- to 7-membered heterocyclic ring; Ar¹ represents an optionally substituted phenyl or pyridyl group; R⁶ represents a hydrogen atom, C₁-C₆ alkyl or is linked to R⁵ as defined above; R⁴ represents a group CHR⁷Ar² or Ar³ or, additionally, in the case where D represents a carbon atom, a group C(O)Ar² or CR⁷(OH)Ar²; Ar² represents an aryl or heteroaryl group which may be optionally substituted; Ar³ represents an acenaphthenyl, indanyl or fluorenyl group, each of which may be optionally substituted; and R⁷ represents a hydrogen atom or a C₁-C₄ alkyl group; or a pharmaceutically-acceptable salt or solvate thereof.

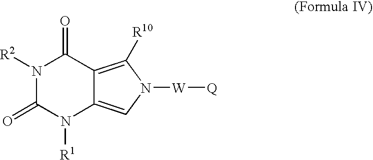

wherein:

W represents —CH₂— or a bond; Q represents Ar¹ or Ar²; in the case where W represents —CH₂—, Q represents an aryl group Ar¹ wherein Ar¹ represents naphthyl, phenyl, quinolyl, isoquinolyl, indolyl, benzofuranyl or benzothienyl; in the case where W represents a bond, Q represents an aryl group Ar² wherein Ar² represents acenaphthenyl, fluorenyl or indanyl; wherein the ring systems which Ar¹ and Ar² represent may all be optionally substituted by one or more substituents selected from C₁₋₄ alkyl, C₁₋₄ alkoxy, halogen, or trifluoromethyl; R¹⁰ represents X—(A)ₚ—Y; X represents S(O)ₙ, C≡C, (CH₂)₂, CH═CH or CH₂CH═CH; n represents 0, 1 or 2; A represents C₁₋₆ alkylene; p is 0 or 1; Y represents CN, OR¹¹, CO₂R¹², CONR¹³R¹⁴, NR¹⁵R¹⁶, NHSO₂R¹⁷, NHCOR¹⁸ or an optionally substituted aryl or heteroaryl group, provided that when X represents S(O)ₙ, and Y is other than an optionally substituted aryl or heteroaryl group, then p is 1 and also provided that when X represents S(O)ₙ, p is 1 and Y represents OH, then n is not 0; R¹³ and R¹⁴ independently represent H, C₁₋₅ alkyl or phenyl, which latter group may be substituted by one or more substituents selected from C₁₋₄ alkyl, C₁₋₄ alkoxy, halogen, or CO₂R²¹; and R¹, R², R¹¹, R¹², R¹⁵, R¹⁶, R¹⁷, R¹⁸ and R²¹ independently represent H or C₁₋₅ alkyl; or a pharmaceutically acceptable derivative thereof.

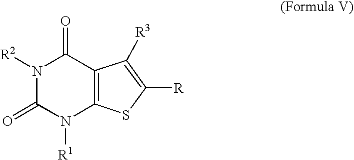

wherein:

R represents a group —C(O)Ar¹ or —C(R⁴)(R⁵)Ar¹;

Ar¹ represents a heterocyclic group comprising a total of from 5 to 10 atoms which include from 1 to 3 heteroatoms independently selected from nitrogen, oxygen and sulfur, which group Ar¹ may be optionally substituted by one or more substituents independently selected from oxo, hydroxyl, C₁₋₄ alkyl, C₁₋₄ alkoxy, halogen, trifluoromethyl, amino, nitro, cyano, trifluoromethoxy, phenoxy, —CH₂N(R⁶)₂, —NHSO₂CF₃, C₁₋₄alkylsulfonylamino, —NHC(O)R^{6a}, CO₂R⁷ or —C(O)NR⁸R^{8a}, with the proviso that Ar¹ does not represent an optionally substituted benzofuranyl, benzothienyl, indolyl, quinolyl or isoquinolyl group;

R⁴represents a hydrogen atom or a C₁₋₄ alkyl group;

R⁵ represents a hydrogen atom or a hydroxyl group;

each R⁶ independently represents a hydrogen atom or a C₁₋₄ alkyl group;

R^{6a} represents a hydrogen atom or a C₁₋₆ alkyl, aryl or arC₁₋₄alkyl group, wherein the aryl group or aryl moiety in the aralkyl group is phenyl or pyridinyl, each of which may be optionally substituted by one or more substituents independently selected from C₁₋₄ alkyl, C₁₋₄ alkoxy, C₁₋₄ alkylcarbonylamino, halogen or trifluoromethyl;

R⁷ represents a hydrogen atom or a C₁₋₄ alkyl group;

R⁸ and R^{8a} each independently represent a hydrogen atom or a C₁₋₄ alkyl, phenyl or pyridinyl group;

R¹ and R² each independently represent a hydrogen atom or a C₁₋₆ alkyl, C₃₋₆ alkenyl, CH₂C₃₋₅ cycloalkyl or C₃₋₆ cycloalkyl group;

R³ represents a hydrogen atom or a group X—R⁹ or X—Ar²;

X represents an oxygen atom, S(O)ₙ, C(O)NR¹⁰, C(O)O, NH(CO)NR¹⁰, NH(CO)O or SO₂NR¹⁰, with the proviso that when X represents an oxygen atom and R represents a group —C(R⁴)(R⁵)Ar¹, then R⁴ and R⁵ both represent a hydrogen atom;

n is 0, 1 or 2;

R⁹ represents a methyl group optionally substituted by one or more substituents independently selected from cyano, carboxyl, C₁₋₅ alkoxycarbonyl, 5-tetrazolyl or C(O)NR¹¹R¹², or R⁹ represents a C₂₋₆ alkyl or C₃₋₆ alkenyl group, each of which may be optionally substituted by one or more substituents independently selected from hydroxyl, cyano, carboxyl, C₁₋₅ alkoxy, C₁₋₅ alkoxycarbonyl, 5-tetrazolyl, azido, phthalimido, SO₂NH₂, C(O)NR¹¹R¹², NR¹³R¹⁴, NHC(O)R¹⁵ or NHSO₂R¹⁶ where R¹¹, R¹², R¹³ and R¹⁴ each independently represent a hydrogen atom or a C₁₋₄ alkyl group, R¹⁵ represents a C₁₋₄ alkyl, C₁₋₄ alkoxy, amino or (di)C₁₋₄alkylamino group or an alkoxyalkylene group containing up to 6 carbon atoms, and R¹⁶ represents a C₁₋₄ alkyl or trifluoromethyl group; or, additionally, in the case where X represents C(O)NR¹⁰, NH(CO)NR¹⁰ or SO₂NR¹⁰, R⁹ and R¹⁰ together with the nitrogen atom to which they are attached may form a 4- to 7-membered saturated heterocyclic ring which may be optionally substituted by one or more hydroxyl groups;

R¹⁰ represents a hydrogen atom or a C₁₋₆ alkyl group or is linked to R⁹ as defined above; and Ar² is phenyl, pyridinyl, thienyl, pyridone or pyridine N-oxide, each of which may be optionally substituted by one or more substituents independently selected from halogen, hydroxyl, nitro, amino, NHSO₂CF₃, C₁₋₄ alkyl, C₁₋₄ alkoxy, bis-C₁₋₄alkanesulfonylamino, C₁₋₄alkylcarbonylamino or C₁₋₄alkoxycarbonylamino;

or a pharmaceutically-acceptable salt or solvate thereof.

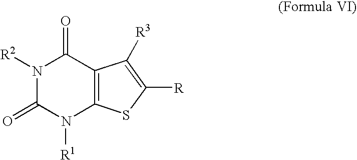

wherein:

R is —C(O)Ar¹, —C(R⁴)(R⁵)Ar¹ or Ar³;

Ar¹ represents a 5- to 10-membered aromatic ring system wherein up to 3 ring atoms may be heteroatoms independently selected from nitrogen, oxygen and sulphur, the ring system being optionally substituted by one or more substituents independently selected from C₁₋₄ alkyl, C₁₋₄ alkoxy, halogen, trifluoromethyl, oxo, nitro, cyano, NR⁶R⁷ and —CH₂NR⁸R⁹; R¹ and R² each independently represent a hydrogen atom, C₁₋₆ alkyl, C₃₋₆ alkenyl, CH₂C₃₋₅ cycloalkyl or C₃₋₆ cycloalkyl;

R³ represents a group X—Ar²;

X represents a group S(O)ₙ, C(O) or CH(OH);

n is 0, 1 or 2;

Ar² represents a 5- or 6-membered aromatic ring wherein up to 4 ring atoms may be heteroatoms independently selected from nitrogen, oxygen and sulphur, the ring being optionally substituted by one or more substituents independently selected from C₁₋₄ alkyl, C₁₋₄ alkoxy, C₁₋₄ alkylthio, halogen, trifluoromethyl, oxo, hydroxyl, amino, nitro, cyano and benzyl;

R⁴ represents a hydrogen atom or C₁₋₄ alkyl;

R⁵represents a hydrogen atom or hydroxyl group;

R⁶ and R⁷ each independently represent a hydrogen atom or C₁₋₄ alkyl, or together with the nitrogen atom to which they are attached form a 5- to 7-membered saturated heterocyclic ring;

R⁸ and R⁹ each independently represent a hydrogen atom or C₁₋₄ alkyl, or together with the nitrogen atom to which they are attached form a 5- to 7-membered saturated heterocyclic ring; and

Ar³ represents acenaphthenyl, indanyl or fluorenyl, each of which may be optionally substituted by one or more substituents independently selected from C₁₋₄ alkyl, C₁₋₄ alkoxy, halogen or trifluoromethyl;

with the proviso that when X represents S(O)ₙ, then Ar² does not represent pyridyl or thienyl; or a pharmaceutically acceptable salt or solvate thereof.

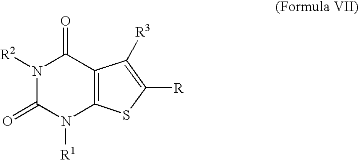

wherein:

R is —C(O)Ar¹, —C(R⁴)(R⁵)Ar¹ or Ar³;

Ar¹ represents a 5- to 10-membered aromatic ring system wherein up to 3 ring atoms may be heteroatoms independently selected from nitrogen, oxygen and sulphur, the ring system being optionally substituted by one or more substituents independently selected from C₁₋₄ alkyl, C₁₋₄ alkoxy, halogen, trifluoromethyl, oxo, nitro, cyano, NR⁶R⁷ and —CH₂NR⁸R⁹;

R¹ and R² each independently represent a hydrogen atom, C₁₋₆ alkyl, C₃₋₆ alkenyl, CH₂C₃₋₅ cycloalkyl or C₃₋₆ cycloalkyl;

R³ represents a group X—R¹⁰ or Ar²;

X represents a bond or a group NR¹¹;

Ar² represents a 5- or 6-membered aromatic ring wherein up to 4 ring atoms may be heteroatoms independently selected from nitrogen, oxygen and sulphur, the ring being optionally substituted by one or more substituents independently selected from C₁₋₄ alkyl, C₁₋₄ alkoxy, C₁₋₄ alkylthio, acetyl, halogen, trifluoromethyl, oxo, hydroxyl, amino, nitro, cyano and benzyl;

R⁴ represents a hydrogen atom or C₁₋₄ alkyl;

R⁵ represents a hydrogen atom or hydroxyl group;

R⁶ and R⁷ each independently represent a hydrogen atom or C₁₋₄ alkyl, or together with the nitrogen atom to which they are attached form a 5- to 7-membered saturated heterocyclic ring;

R⁸ and R⁹ each independently represent a hydrogen atom or C₁₋₄ alkyl, or together with the nitrogen atom to which they are attached form a 5- to 7-membered saturated heterocyclic ring;

R¹⁰ represents C₁₋₆ alkyl, C₂₋₆ alkenyl or C₂₋₆ alkynyl, each of which may be optionally subsituted by one or more substituents independently selected from carboxyl, hydroxyl, —C(O)—R¹², C₃₋₆ cycloalkyl, morpholinyl, —NR¹³R¹⁴, —SR¹⁵, —OR¹⁶, phenyl and halophenyl, or

R¹⁰ represents a C₃₋₆ cycloalkylcarbonyl, —C(O)CH₂CN, halophenylcarbonyl or trifluoromethylcarbonyl group;

R¹¹ represents a hydrogen atom or a C₁₋₆ alkyl group;

R¹² represents piperazinyl optionally substituted by a C₁₋₆ alkyl group, or R¹² represents a group —NR¹⁷R⁸;

R¹³ and R¹⁴ each independently represent a hydrogen atom, or a C₁₋₄ alkyl, C₁₋₄ hydroxyalkyl or —C(O)—R¹⁹ group, or

R¹³ and R¹⁴, together with the nitrogen atom to which they are attached, form a 5- to 7-membered saturated heterocyclic ring which may be optionally substituted by one or more substituents independently selected from C₁₋₄ alkyl, hydroxyl and oxo;

R¹⁵ and R¹⁶ each independently represent a 5- or 6-membered aromatic ring wherein up to 4 ring atoms may be heteroatoms independently selected from nitrogen, oxygen and sulphur, the ring being optionally substituted by one or more substituents independently selected from halogen atoms, cyano and C₁₋₄ alkyl;

R¹⁷ and R¹⁸ each independently represent a hydrogen atom, or a C₁₋₄ alkyl group optionally substituted by one or more substituents independently selected from halogen atoms and hydroxyl;

R¹⁹ represents a C₁₋₆ alkyl or C₃₋₆ cycloalkyl group, each of which may be optionally substituted by a hydroxyl group; and

Ar³ represents acenaphthenyl, indanyl or fluorenyl, each of which may be optionally substituted by one or more substituents independently selected from C₁₋₄ alkyl, C₁₋₄ alkoxy, halogen and trifluoromethyl;

or a pharmaceutically acceptable salt or solvate thereof.

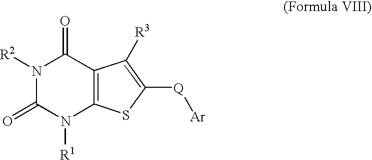

wherein:

R¹ and R² each independently represent a C₁₋₆alkyl, C₃₋₆alkenyl, C₃₋₅cycloalkyl(C₁₋₃)methyl or C₃₋₆cycloalkyl; each of which may be optionally substituted by 1 to 3 halogen atoms;

R³ represents a group —CON(R¹⁰)YR¹¹ or —SO₂N(R¹⁰)YR¹¹;

[wherein Y is O, S or NR₁₂ (wherein R¹² is hydrogen or C₁₋₆alkyl);

and R¹⁰ and R¹¹ are independently C₁₋₆alkyl optionally substituted by halo, hydroxy, amino, C₁₋₆alkylamino or di-(C₁₋₆alkyl)amino];

Q is —CO— or —C(R⁴)(R⁵)— (wherein R⁴ represents a hydrogen atom or C₁₋₄alkyl and R⁵ represents a hydrogen atom or hydroxyl group);

Ar represents a 5- to 10-membered aromatic ring system wherein up to 4 ring atoms may be heteroatoms independently selected from nitrogen, oxygen and sulphur, the ring system being optionally substituted by one or more substituents independently selected from C₁₋₄alkyl, C₁₋₄alkoxy, halogen, haloalkyl, dihaloalkyl, trihaloalkyl, hydroxyC₁₋₄alkyl, C₁₋₄alkoxyC₁₋₄alkyl, C₁₋₄alkylthio, C₁₋₄alkoxycarbonyl, C₂₋₄alkanoyl, oxo, nitro, cyano, —N(R⁶)R⁷ and —(CH₂)ₚN(R⁸)R⁹, hydroxy, C₁₋₄alkylsulphonyl, C₁₋₄alkylsulphinyl, carbamoyl, C₁₋₄alkylcarbamoyl, di-(C₁₋₄alkyl)carbamoyl, carboxy;

p is 1 to 4;

R⁶ and R⁷ each independently represent a hydrogen atom, C₁₋₄alkanoyl or C₁₋₄alkyl, or together with the nitrogen atom to which they are attached form a 5- to 7-membered saturated heterocyclic ring;

R⁸ and R⁹ each independently represent a hydrogen atom, C₁₋₄alkanoyl or C₁₋₄ alkyl, or together with the nitrogen atom to which they are attached form a 5- to 7-membered saturated heterocyclic ring;

or a pharmaceutically acceptable salt or prodrug thereof.

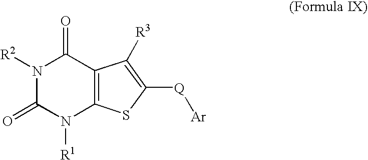

wherein:

R¹ and R² each independently represent a C₁₋₆alkyl, C₃₋₆alkenyl, C₃₋₅cycloalkyl(C₁₋₃)methyl or C₃₋₆cycloalkyl; each of which may be optionally substituted by 1 to 3 halogen atoms;

R³ is isoxazolidin-2-ylcarbonyl or tetrahydroisoxazin-2-ylcarbonyl wherein each ring is optionally substituted by one hydroxy group;

Q is —CO— or —C(R⁴)(R⁵)— (wherein R⁴ represents a hydrogen atom or C₁₋₄alkyl and R⁵ represents a hydrogen atom or hydroxyl group);

Ar represents a 5- to 10-membered aromatic ring system wherein up to 4 ring atoms may be heteroatoms independently selected from nitrogen, oxygen and sulphur, the ring system being optionally substituted by one or more substituents independently selected from C₁₋₄alkyl (optionally substituted by 1,2 or 3 hydroxy groups), C₁₋₄alkoxy, halogen, haloalkyl, dihaloalkyl, trihaloalkyl, C₁₋₄alkoxyC₁₋₄alkyl, C₁₋₄alkylthio, C₁₋₄alkoxycarbonyl, C₂₋₄alkanoyl, oxo, thioxo, nitro, cyano, —N(R⁶)R⁷ and —(CH₂)ₚN(R⁸)R⁹, hydroxy, C₁₋₄alkylsulphonyl, C₁₋₄alkylsulphinyl, carbamoyl, C₁₋₄alkylcarbamoyl, di-(C₁₋₄alkyl)carbamoyl, carboxy;

p is 1 to 4;

R⁶ and R⁷ each independently represent a hydrogen atom, C₁₋₄alkanoyl or C₁₋₄alkyl, or together with the nitrogen atom to which they are attached form a 5- to 7-membered saturated heterocyclic ring;

R⁸ and R⁹ each independently represent a hydrogen atom, C₁₋₄alkanoyl or C₁₋₄ alkyl, or together with the nitrogen atom to which they are attached form a 5- to 7-membered saturated heterocyclic ring;

or a pharmaceutically acceptable salt or prodrug thereof.

At the filing dates of these applications the mechanism of action of these compounds was not known and therefore is not taught in these applications/publications. Moreover, the newly identified mechanism of action is distinct from, indeed a radical change, from that acted on by current immunosuppressive pharmaceutical compounds, such as cyclosporin A.

The medical treatment methods of the invention are particularly suitable for treating rheumatoid arthritis and for use before, during and after transplantation surgery, to prevent host rejection of the transplanted tissue. Cancers treated using the methods of the invention are preferably not CNS cancers such as glial cell cancers or cancers that have metastasized to brain.

Compounds capable of effecting monocarboxylate build up in a cell or preventing monocarboxylate efflux from the cell are particularly suitable for use in the disease treatment methods of the invention.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder), or for parenteral administration (for example as a sterile aqueous or oily solution for intravenous, subcutaneous, intramuscular or intraperitoneal dosing or as a suppository for rectal dosing). It may be desirable to administer the therapeutic agent directly to or at the site of interest, e.g., injection into arthritic joint. This may, for example, obviate any non-specific cellular side effects that the agent may cause.

The compositions of the invention may be obtained by conventional procedures using conventional pharmaceutical excipients, well known in the art. Thus, compositions intended for oral use may contain, for example, one or more colouring, sweetening, flavouring and/or preservative agents.

In addition to the compounds of the present invention the pharmaceutical composition of this invention may also contain, or be co-administered (simultaneously or sequentially) with, one or more pharmacological agents of value in treating one or more disease conditions referred to herein, for example, agents such as FK506, Cyclosporin A, steroids, azathioprine, mycophenolate mofetil, leflunomide, methotrexate and antibodies against TNF and its receptor.

The pharmaceutical compositions of this invention will normally be administered to a warm-blooded animal at a unit dose within the range 5-5000 mg per square meter body area of the animal, i.e., approximately 0.1-100 mg/kg, and this normally provides a therapeutically-effective dose. A unit dose form such as a tablet or capsule will usually contain, for example 1-250 mg of active ingredient. Preferably, a daily dose in the range of 1-50 mg/kg is employed. In general, lower doses will be administered when a parenteral route is employed. Thus, for intravenous administration, a dose in the range of, for example, 0.5 mg to 30 mg per kg body weight will generally be used. Similarly, for administration by inhalation, a dose in the range of, for example, 0.5 mg to 25 mg per kg body weight will be used. Oral administration is preferred, particularly in tablet form. Typically, unit dosage forms will contain about 1 mg to 500 mg of a compound of this invention. However, the size of the dose for therapeutic or prophylactic purposes will naturally vary according to the nature and severity of the conditions, the age and sex of the animal or patient and the route of administration, according to well known principles of medicine. Accordingly, the practitioner who is treating a particular patient may determine the optimal dosage. A therapeutically effective dose or amount refers to that amount of the agent sufficient to prevent development of or to alleviate the existing symptoms associated with the disorder. Determination of the effective amounts is well within the capabilities of those skilled in the art, especially in light of the detailed disclosure herein. For example, the therapeutically effective amount can be estimated initially from cell culture assays. A dose can be formulated in animal models to achieve a circulating concentration range that includes the IC50 (concentration at which 50% of the maximal effect is demonstrated) as determined in in vitro cellular assays. Such information can be used to more accurately determine the therapeutically effective dose in humans.

The amount of active ingredient that is combined with one or more excipients to produce a single dosage form will necessarily vary depending upon the host treated and the particular route of administration. A formulation intended for oral administration to humans will generally contain, for example, from 0.5 mg to 0.5 g of active agent compounded with an appropriate and convenient amount of excipients, which may vary from about 5 to about 98 percent by weight of the total composition.

Techniques for formulation and administration of agents for use in accordance with the present invention may be found in the latest edition of “Remington's Pharmaceutical Sciences”, Mack Publishing Co., Easton, Pa.

According to one aspect of the invention, there is provided the use of a compound capable of inhibiting cellular monocarboxylate transport, or a pharmaceutically-acceptable composition thereof, in the manufacture of a medicament for use in the production of an anti-proliferative effect in a warm-blooded animal, such as man. An anti-proliferative effect is defined herein as the ability to prevent cell number expansion. In one embodiment the compound is a selective inhibitor; in another embodiment it has broad spectrum activity.

According to a further feature of this aspect of the invention there is provided a method for producing an anti-proliferative effect in a warm-blooded animal, such as man, in need of such treatment, which method comprises administering to said animal an effective amount of a compound capable of inhibiting monocarboxylate transport within a cell, or a pharmaceutically-acceptable composition thereof, as defined hereinbefore. Again, in one embodiment the compound is a selective inhibitor, while in another embodiment it has broad spectrum activity.

A variety of gene therapy approaches may be used in accordance with the invention to modulate expression of an MCT gene in vivo. One therapeutic means of inhibiting or dampening the expression levels of a particular gene (for example one of the MCT proteins) is to use antisense therapy. Antisense therapy utilises antisense nucleic acid molecules that are synthetic segments of DNA or RNA (“oligonucleotides”), designed to mirror specific mRNA sequences and block protein production by inhibiting translation of the native gene transcript. Once formed, the mRNA binds to a ribosome, the cell's protein production “factory” which effectively reads the RNA sequence and manufactures the specific protein molecule dictated by the gene. If an antisense molecule is delivered to the cell (for example as native oligonucleotide or via a suitable antisense expression vector), it binds to the messenger RNA because its sequence is designed to be a complement of the target sequence of bases. Once the two strands bind, the mRNA may no longer dictate the manufacture of the encoded protein by the ribosome and/or is rapidly broken down by the cell's enzymes (e.g., RNaseH), thereby freeing the antisense oligonucleotide to seek and disable another identical messenger strand of mRNA. Oligonucleotides that are complementary to and hybridisable with any portion of MCT mRNA are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from previously known polynucleotides are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. It is preferred that the sequence be at least 17 nucleotides in length in order to achieve sufficiently strong annealing to the target mRNA sequence to prevent translation. Antisense oligonucleotides targeting MCT1 (in Caco-2 cells) have been described by Hadjiagapiou et al. (Am. J. Physiol. Gastrointest. Liver Physiol. 279:G775-G780, 2000). Antisense molecules may be introduced into cells by microinjection, liposome encapsulation or by expression from vectors harboring the antisense sequence.

Alternatively, ribozyme molecules may be designed to cleave and destroy the MCT mRNAs in vivo. Ribozymes are RNA molecules that possess highly specific endoribonuclease activity. Hammerhead ribozymes comprise a hybridising region that is complementary in nucleotide sequence to at least part of the target RNA, and a catalytic region that is adapted to recognise and cleave the target RNA. The hybridising region preferably contains at least 9 nucleotides. The design, construction and use of such ribozymes are well known in the art and are more fully described in Haselhoff and Gerlach (Nature 334:585-591, 1988). In another alternative, oligonucleotides designed to hybridise to the 5′-region of the MCT gene so as to form triple helix structures may be used to block or reduce transcription of the MCT gene. In yet another alternative, RNA interference (RNAi) oligonucleotides or short (20-25 bp) RNAi MCT sequences cloned into plasmid vectors are designed to introduce double stranded RNA into mammalian cells to inhibit and/or result in the degradation of MCT messenger RNA. MCT RNAi molecules may begin with adenine/adenine (AA). They may be 20, 21, 22, 23, 24 or 25 base pair double stranded RNA molecules with the preferred length being 21 base pairs and 2-nucleotide 3′ overhangs, alternatively they may be hairpin forming 45-50 mer RNA molecules. They would be specific for MCT mRNA. The design, construction and use of such molecules is well known in the art and is more fully described in Elbashir et al. (Nature. 411(6836):428-429, 2001). In one embodiment, the antisense, ribozyme, triple helix or RNAi nucleotides are designed to specifically inhibit translation and/or transcription of only one MCT, with minimal effects on the other MCT genes.

Thus, according to another aspect of the invention there is provided a method for treating a patient suffering from an immune-mediated disorder or cancer (particularly cancer other than a CNS cancer such as glial cell cancer), comprising identifying a patient in need of such treatment and administering to said patient an effective amount of an anti-sense molecule, a ribozyme molecule, triple helix forming molecule or RNAi molecule capable of binding to the mRNA of an MCT, as hereinbefore described, including any nucleic acid or protein derived inhibitor of transcription or translation of MCTs.

There is also provided the use of an antisense nucleic acid molecule, a ribozyme molecule, a triple helix forming molecule, RNAi molecule or an antibody directed against an MCT, in the treatment of, or manufacture of a medicament for treating, a cell proliferative disorder.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention is further described, but in no way limited, by the following examples:

### EXAMPLE 1

### Design and Construction of Ligands

1A Ligand A

i) 2,6-Dihydro-6-[(2-iodophenyl)methyl]-2-methyl-4-(2-methylpropyl)-1H-pyrrolo[3,4-d]pyridazin-1-one

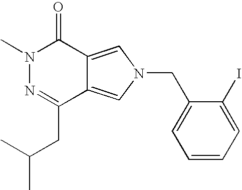

2,6-Dihydro-2-methyl-4-(2-methylpropyl)-1H-pyrrolo[3,4-d]pyridazin-1-one (205 mg), 1-chloromethyl-2-iodo-benzene (300 mg) [see WO99/29695], and caesium carbonate (360 mg) were mixed in dry DMF (1.5 ml). After stirring at room temperature for 2.5 hr under nitrogen the reaction was evaporated to dryness, and the residue was partitioned between ethyl acetate and dilute HCl. The organic solution was washed with brine, dried and evaporated. The residue was purified by chromatography to give a solid, which was recrystallised from cyclohexane/ethyl acetate to afford the sub-title compound 270 mg.

MS (APCI+ve) (M+H)⁺422 NMR ¹H δ_{(CDCl3)} 0.96(6H, d), 2.14(1H, m), 2.56(2H, d), 3.72(3H, s), 5.32(2H, s), 6.86(1H, d), 7.05(1H, d), 7.06(1H, t), 7.33(1H, t), 7.47(1H, d), 7.90(1H, d).

ii) 7-[[3-[[(1,1-Ddimethylethyl)dimethylsilyl]oxy]propyl]thio]-2,6-dihydro-6-[(2-iodophenyl)methyl]-2-methyl-4-(2-methylpropyl)-1H-pyrrolo[3,4-d]pyridazin-1-one

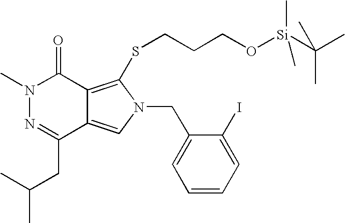

The product of step (i) (1.1 g) and S-[3-[[(1,1-dimethylethyl)dimethylsilyl]propyloxy]-1-(4-methylphenyl)dioxidosulfanyl-propanethiol 4-methyl-benzenesulfonothioate (1.7 g) were combined in THF(40 ml) under nitrogen at −78° C., and a solution of lithium diisopropylamide (0.6M, 8.3 ml) was added dropwise. The reaction was quenched with saturated aqueous sodium bicarbonate solution after 2 hr at −78° C. and then the mixture was extracted into ethyl acetate. The organic solution was washed with brine, dried and evaporated. The residue was purified by chromatography to afford the sub-title compound (400 mg).

NMR ¹H δ_{(CDCl3)} 0.0(6H, s), 0.84(9H, s), 0.95(6H, d), 1.70(2H, m), 2.08(1H, m), 2.52(2H, d), 3.09(2H, t), 3.60(2H, t), 3.74(3H, s), 5.47(2H, s), 6.40(1H, d), 6.99(1H, t), 7.07(1H, s), 7.25(1H, t), 7.90(1H, d).

iii) 7-[[3-[[(1,1-Ddimethylethyl)dimethylsilyl]oxy]propyl]thio]-2,6-dihydro-2-methyl-4-2-methylpropyl)-6-[[2-(trimethylstannyl)phenyl]methyl]-1H-pyrrolo[3,4-d]pyridazin-1-one

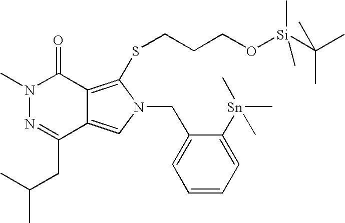

The product of step (ii) (34 mg) in dry toluene (1 ml) was degassed by purging with nitrogen, and then hexamethyl ditin (100 microlitre) and tetrakistriphenylphosphine palladium (0) (10 mg) were added. The mixture was heated and stirred under nitrogen in a sealed flask at 95° C. for 4 hr. The reaction was cooled, diluted with ethyl acetate and filtered through a pad of silica, and then purified by chromatography to afford the sub-title compound (20 mg).

NMR ¹H δ_{(CDCl3)} 0.0(6H, s), 0.4(9H, s+Sn satellites at 0.32 and 0.49), 0.86(9H, s), 0.96(6H, d), 1.70(2H, m), 2.08(1H, m), 2.52(2H, d), 3.06(2H, t), 3.58(2H, t), 3.75(3H, s), 5.50(2H, s), 6.54(1H, d), 6.98(1H, t), 7.25-7.29(2H, m), 7.54(1H, d).

iv) 2,6-Ddihydro-7-[(3-hydroxypropyl)thio]-2-methyl-4-(2-methylpropyl)-6-[[2-(trimethylstannyl)phenyl]methyl]-1H-pyrrolo[3,4-d]pyridazin-1-one

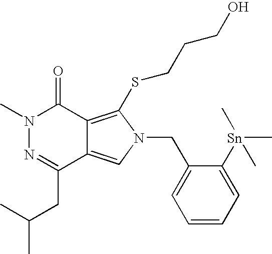

The product of step (iii) (6 mg) was treated with a 1M solution of TBAF in THF (0.5 ml). After 1.5 hr the reaction was diluted with saturated aqueous sodium bicarbonate solution, and then extracted into ethyl acetate. The organic solution was washed with brine, dried and evaporated. Chromatography of the residue gave the sub-title compound (3 mg).

NMR ¹H δ_{(CDCl3)} 0.39(9H, s+Sn satellites at 0.33 and 0.46), 0.94(6H, d), 1.73(2H, m), 2.08(1H, m), 2.52(2H, d), 3.00(2H, t), 3.74(3H, s), 3.80(3H, m), 5.50(2H, s), 6.54(1H, d), 6.50(1H, d), 7.01(1H, s), 7.21-7.29(2H, m), 7.54(1H, d).

v) 2,6-Dihydro-7-[(3-hydroxypropyl)thio]-6-[(2-[¹²⁵I]iodophenyl)methyl]-2-methyl-4-(2-methylpropyl)-1H-pyrrolo[3,4-d]pyridazin-1-one

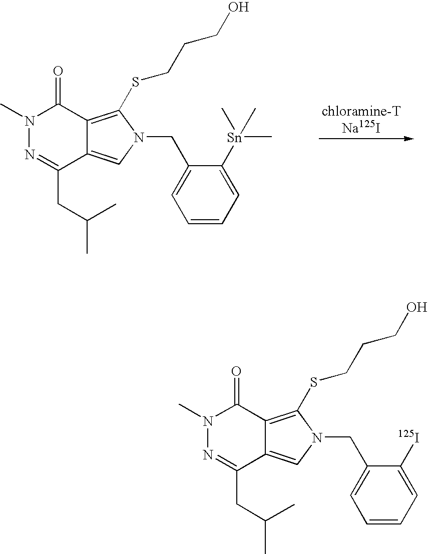

To a solution of sodium [¹²⁵I]iodide (Amersham Pharmacia Biotech; ≈2000 Ci mmol⁻¹, 1 mCi; 0.5 nmol, 10 μl) was added a solution of the product of step (iv) in methanol (10 μl, 3.65 nmol; 200 μg ml⁻¹, 365 nmol ml⁻¹) followed by chloramine-T in water (4 μl, 8.8 nmol; 50 μg ml⁻¹, 2192 nmol ml⁻¹). The vial was sealed, shaken vigorously and left to stand at room temperature for 30 min.

The product was purified by preparative HPLC. The radiochemical purity was typically >98%. The radioactive concentration was determined by liquid scintillation counting and was normally found to be in the range of 5 to 8 MBq ml⁻¹. The radiochemical yield was typically between 50-60%.

vi) 2,6-Dihydro-7-[(3-hydroxypropyl)thio]-6-[(2-iodophenyl)methyl]-2-methyl-4-(2-methylpropyl)-1H-pyrrolo[3,4-d]pyridazin-1-one

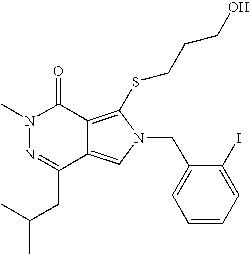

(Cold Ligand A)

The title compound was prepared by the method of step (iv) using the product of step (ii).

MS (APCI+ve) (M+H)⁺512 NMR ¹H δ_{(CDCl3)} 0.95(6H, d), 1.77 (2H, quintet), 2.10 (1H, septet), 2.54 (2H, d), 3.08 (2H, t), 3.74 (3H, s), 3.86-3.94 (3H, m), 5.50 (2H, s), 6.37-6.41 (1H, m), 6.99-7.06 (1H, m), 7.10 (1H, d), 7.22-7.28 (1H, m), 7.91 (1H, d).

1B Ligand B

i) 1-Azido-4-chloromethyl-2-iodo-benzene

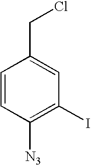

4-Azido-3-iodo-benzenemethanol (J. Labelled Compd. Radiopharm., 1996, 38:227-37) (350 mg) in dry dichloromethane (20 ml) was treated with triethylamine (185 microlitre) and methanesulphonyl chloride (100 microlitre) at room temperature for 20 hr. Volatiles were removed in vacuo and the residue was purified by chromatography to give the sub-title compound 210 mg.

NMR ¹H δ_{(CDCl3)} 4.51(2H, s), 7.11(1H, d), 7.42(1H, dd), 7.82(1H, d).

ii) 6-[(4-Azido-3-iodophenyl)methyl]-2,6-dihydro-2-methyl-4-(2-methylpropyl)-1H-pyrrolo[3,4-d]pyridazin-1-one (unlabelled ligand B)

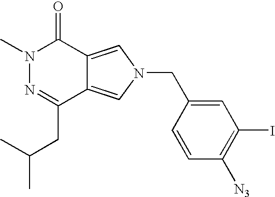

2,6-Dihydro-2-methyl-4-(2-methylpropyl)-1H-pyrrolo[3,4-d]pyridazin-1-one (WO99/29695) (25 mg), the product of step (i) (40 mg) and caesium carbonate (40 mg) were mixed in dry DMF (5 ml). After stirring at room temperature for 3 days under nitrogen the reaction was poured into water and extracted into ethyl acetate. The organic solution was washed with brine, dried and evaporated. The residue was purified by chromatography to give the sub-title compound 50 mg.

MS (APCI+ve) (M+H)⁺463 MP 138-9° C. NMR ¹H δ_{(CDCl3)} 0.97 (6H, d), 2.06-2.20 (1H, m), 2.55 (2H, d), 3.72 (3H, s), 5.20 (2H, s), 6.98 (1H, d), 7.11 (1H, d), 7.17 (1H, dd), 7.47 (1H, d), 7.62 (1H, d).

iii) 6-[(4-Azido-3-(trimethylstannyl)phenyl)methyl]-2,6-dihydro-2-methyl-4-(2-methylpropyl)-1H-pyrrolo[3,4-d]pyridazin-1-one

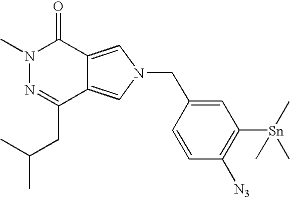

6-[(4-Azido-3-iodophenyl)methyl]-2,6-dihydro-2-methyl-4-(2-methylpropyl)-1H-pyrrolo[3,4-d]pyridazin-1-one (10 mg), hexamethyl ditin (100 μl) and tetrakis triphenylphosphine Pd (0) (2 mg) were combined in dry toluene (5 ml) and heated at 100° C. under nitrogen for 4 hours. After cooling all volatiles were removed in vacuo and the residue was purified by preparative thin layer chromatography (SiO₂/ethyl acetate) to afford the sub-title compound (10 mg).

NMR ¹H δ_{(CDCl3)} 0.3(9H, s+Sn satellites at 0.21 and 0.4, 2×d), 0.96(6H, d), 2.13(1H, m), 2.55(2H, d), 3.72(3H, s), 5.22(2H, s), 7.01(1H, d), 7.10-7.22(3H, m), 7.46(1H, d).

iv) 6-[(4-Azido-3-[¹²⁵I]iodophenyl)methyl]-2,6-dihydro-2-methyl-4-(2-methylpropyl)-1H-pyrrolo[3,4-d]pyridazin-1-one

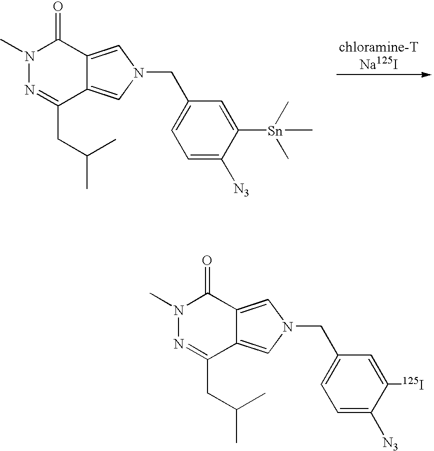

To a solution of sodium [¹²⁵I]iodide (Amersham Pharmacia Biotech, IMS30; ≈2000 Ci mmol⁻¹, 1 mCi; 0.5 nmol, 10 μl) was added a solution of the product of step (iii) (10 μl, 3.0 nmol; 150 μg ml⁻¹, 300 nmol ml⁻¹) followed by chloramine-T in water (2 μl, 3.6 nmol; 500 μg ml⁻¹, 1800 nmol ml⁻¹). The vial was sealed, shaken and left to stand at room temperature for 10 minutes. An aliquot of sodium metabisulphite (2 μl, 16 nmol, 1500 μg ml⁻¹, 8000 nmol ml⁻¹) was added to the reaction followed by methanol (25 μl) and the vial shaken.

The iodo-azide product was purified using preparative HPLC. The radiochemical purity was typically >99%. The radioactive concentration was determined by liquid scintillation counting and was normally found to be in the range of 2 to 3 MBq ml⁻¹. The radiochemical yield was typically between 20-30%.

1C Ligand C

i) 1,2,3,4-Tetrahydro-1-(2-hydroxypropyl)-3,6-dimethyl-2,4-dioxo-5-pyrimidine carbonitrile

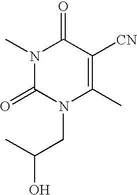

Ethyl N-(2-cyano-3-ethoxy-1-oxo-2-butenyl)-carbamate (Chem. Pharm. Bull, 1972, 20, 1380-8) (5 g) was dissolved in ethanol (50 ml) at reflux under nitrogen, and DL-1-amino-2-propanol (1.88 ml) added. After 5 hr at reflux the reaction was cooled and evaporated to dryness. The resulting gum was suspended in water (50 ml), treated with sodium hydroxide (1.46 g) and stirred 1 hr. Dimethyl sulphate (3.45 ml) was added and stirring was continued for 1 hr. The precipitate was collected, and the aqueous solution was concentrated, then extracted into dichloromethane. The organic solution was dried and evaporated and the residue was combined with the precipitate (above) to afford the subtitle compound (4.35 g).

MS (EI) (M+H)⁺223 BP 159

ii) 1-(2-Hhydroxypropyl)-3-methyl-6-(1-naphthalenylmethyl)-1H-pyrrolo[3,4-d]pyrimidine-2,4(3H,6H)-dione

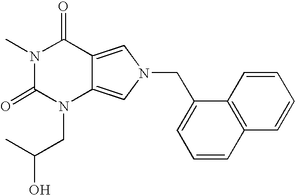

The product of step (i) (4.24 g) was suspended in 75% formic acid (80 ml) and Raney Nickel (50% dispersion in 8 ml water) was added. The mixture was heated at 90° C. under nitrogen for 15 min. After cooling the suspension was filtered (kieselguhr) and evaporated. The residue was dissolved in water (100 ml) and extracted into ethyl acetate, each aliquot of extraction was washed with sodium bicarbonate solution. The combined organic extracts were dried and evaporated to yield a white foam, which was dissolved in chloroform (20 ml) and heated to 50° C. A solution of bromine (0.4 ml) in chloroform (5 ml) was added and after stirring for 10 min at 50° C. was concentrated in vacuo. The residue was dissolved in ethanol (25 ml), treated with triethylamine (2.96 ml) and then 1-naphthalenylmethylamine (1.55 ml) was added. After 20 hr at room temperature the reaction was poured into 2M HCl (100 ml) and extracted with ethyl acetate, dried (MgSO₄) and then concentrated in vacuo. Purification by chromatography (SiO2/2:1 hexane-ethyl acetate) gave the sub-title compound, which was crystallised from hexane/ethyl acetate to afford sub-title compound, 110 mg.

MS (EI) (M+H)⁺ 363 BP 141

iii ) 3-Methyl-6-(1-naphthalenylmethyl)-1-(2-oxopropyl)-1H-pyrrolo[3,4-d]pyrimidine-2,4(3H,6H)-dione

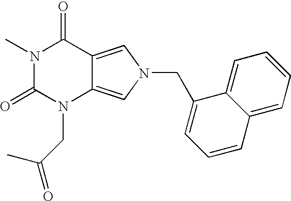

A solution of anhydrous DMSO (417 μl) in anhydrous dichloromethane (10 ml) was added dropwise to a solution of oxalyl chloride (256 μl) in dichloromethane (20 ml) at −78° C. under nitrogen. After 15 min a solution of the product of step (ii) (970 mg) in dichloromethane (20 ml) at −78° C. was added. After 5 min triethylamine (900 μl) was added, the reaction was stirred 10 min then allowed to warm to 0° C. Water (100 ml) was added and the mixture was extracted with dichloromethane. Drying (MgSO₄), evaporation and chromatography gave the sub-title compound (660 mg).

MS (EI) (M+H)⁺ 361 BP 141

iv) 3-Methyl-1-(2-methyl-2-propenyl)-6-(1-naphthalenylmethyl)-1H-pyrrolo[3,4-d]pyrimidine-2,4(3H,6H)-dione

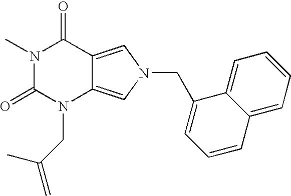

A stirred suspension of methylene triphenylphosphonium bromide (1.22 g) in dry THF (20 ml) at −78° C. under nitrogen was treated with sodium hexamethyldisilazide (3.1 ml of 1M solution in THF). The reaction mixture was stirred at room temperature for 1 hr. The resulting solution was added to a solution of the product of step (iii) (560 mg) in dry THF (30 ml) at 0° C. under nitrogen, and stirred at 5° C. for 2 hr then 20 min at room temperature. The mixture was poured into water (50 ml) and extracted into ethyl acetate. Drying, evaporation and chromatography gave the sub-title compound (465 mg).

MS (EI) (M+H)⁺ 339 BP 141

v) 5-[(3-Hydroxypropyl)thio]-3-methyl-1-(2-methyl-2-propenyl)-6-(1-naphthalenylmethyl)-1H-pyrrolo[3,4-d]pyrimidine-2,4(3H,6H)-dione

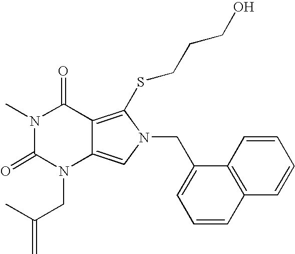

The product of step (iv) (350 mg) and S-[3-[[(1,1-dimethylethyl)dimethylsilyl]propyloxy]-1-(4-methylphenyl)dioxidosulfanyl-propanethiol 4-methyl-benzenesulfonothioate (527 mg) were dissolved in dry THF (10 ml) at −78° C. under nitrogen. LDA (1.95 mmol) in dry THF (5 ml) was added, and after 1 hr the temperature was raised to 0° C. The reaction was quenched by addition of sodium bicarbonate solution (30 ml), and extracted into ether. Drying and evaporation gave a residue which was dissolved in acetonitrile (10 ml) and treated with 40% HF (0.4 ml) for 30 min. The reaction mixture was poured into sodium bicarbonate solution and extracted into ethyl acetate. Drying, evaporation and chromatography gave the sub-title compound which was recrystallised from hexane-ethyl acetate afford the product (163 mg).

NMR ¹H nmr δ_{(CDCl3)} 1.67(3H, s), 1.8(2h, m), 3.1(2H, t), 3.44(3H, s), 3.83(2H, dd), 4.53(2H, s), 4.72(1H, s), 4.83(1H, s), 5.83(2H, s), 6.37(1H, s), 6.76(1H, d), 7.39(1H, t), 7.58(2H, m), 7.83(1H, d), 7.79(2H, m).

vi) 5-[(3-Hydroxypropyl)thio]-1-([2,3,3′-³H]isobutyl)-3-methyl-6-(1-naphthalenylmethyl)-1H-pyrrolo[3,4-d]pyrimidine-2,4(3H,6H)-dione 5-[(3-Hydroxypropyl)thio]-3-methyl-1-[2,3-di³H-2-(³H-methyl)propyl]-6-(1-naphthalenylmethyl)-1H-pyrrolo[3,4-d]pyrimidine-2,4(3H,6H)-dione

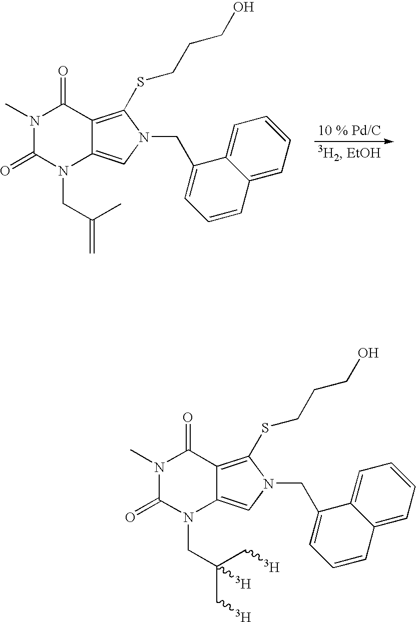

The product of step (v) (2.28 mg, 5.1 μmol), 10% Pd/carbon (2.35 mg) and ethanol (0.5 ml) were placed in a 1 ml round-bottomed flask which was attached to a tritium manifold. The contents of the flask were frozen in liquid nitrogen and the flask then evacuated before tritium gas (241 GBq, 2.6 ml, 0.113 mmol) was introduced. The flask was allowed to warm to room temperature and the contents left to stir for 22 hours.

The flask was removed from the apparatus and the catalyst removed by filtration. The filtrate was diluted with ethanol (5 ml) and the solvent removed under reduced pressure. This was repeated with a further portion of ethanol (5 ml).

Purification of the tritiated material was achieved by reversed-phase HPLC using a Waters Novapak C₁₈ 200×8 mm radial compression module eluting with 45% v/v acetonitrile/0.1% v/v aqueous trifluoroacetic acid at 3 ml min⁻¹ and UV detection at 254 nm. An aliquot of the stock solution (5 ml) was reduced to dryness and re-dissolved in acetonitrile (0.5 ml) and purified in approximately three equal injections. The peak due to ligand C was collected as three separate fractions (front, middle and back) and these were combined with the equivalent cuts from the two subsequent injections. The volumes of the three fractions were measured in each case and made up to 10 ml by the addition of 50% w/v aqueous sodium thiosulphate (100 μl) and ethanol. The radioactive concentration, molar specific activity and radiochemical purity of the three fractions was determined and the details included in the table below.

TABLE 3

| | Radioactive | Molar Specific | Radio- |
|---|---|---|---|
| | Concentration | Activity | chemical |
| Fraction | (MBq ml⁻¹) | (GBq mmol⁻¹) | Purity |
| Front fraction | 24.53 | 1713.1 | 97.8% |
| Middle fraction | 34.78 | 1631.7 | 97.9% |
| Back fraction | 14.39 | 1217.3 | ≈78% |

Both the front and middle fractions provided material of a suitable radiochemical purity and specific activity for use in the ligand binding assay.

Unlabelled ligand C is disclosed in Michne et al. (J Med. Chem. (1995) 38:2557-2569).

### EXAMPLE 2

### Photoaffinity Labelling of the Target Protein

The unlabelled compounds and radioligands of Example 1 were used to identify the precise target(s) involved in the binding interaction using photoaffinity labelling, gel electrophoresis, peptide sequencing and mass spectrometry. The target protein was identified as being MCT1.

Photoaffinity labelling was performed using ¹²⁵I-labelled ligand B. Photolabelling reactions were set up by diluting washed rat red-blood cell membranes 10-fold in assay buffer (50 mM HEPES (pH 7.5); 0.1 mM EDTA; 150 mM NaCl) and by incubating in the presence or absence of 1 μM unlabelled competing ligand C. The ¹²⁵I-photoligand solution was added to give a final ligand concentration of 1 nM. The photolabelling reaction was performed by irradiating the sample with a hand held 254 nm UV source for 1 minute at room temperature. The labelled ghost membranes were then collected by centrifugation at 100,000 g for 10 minutes at 4° C. Finally, the samples were each washed in 1 ml of distilled water and the final membrane pellets were collected at 100,000 g for 10 minutes at 4° C. The samples were stored at −20° C. until use.

The photoaffinity-labelled proteins were analysed by one- and two-dimensional gel electrophoresis. The labelled proteins were excised from the gel and subjected to in gel protein digestion prior to analyses by mass spectrometry. Three peptides matching rat monocarboxylate transporter 1 were identified.

### EXAMPLE 3

### Cloning of MCT Genes

3.1 Human MCT1

Oligonucleotide primers containing unique restriction sites, to allow subsequent cloning, and sequences derived from the optimal Kozak consensus sequence (MCT1-5′; 5′-GGA-TCC-ACC-ATG-CCA-CCA-GCA-GTT-GGA-GG-3′; SEQ ID No: 41; and MCT1-3′; 5′-GTC-GAC-TCA-GAC-TGG-ACT-TTC-CTC-CTC-CTT-G-3′; SEQ ID No: 42) were used in a PCR to amplify the MCT1 ORF from a cDNA library. The PCR fragment was subcloned into the vector pCR3.1 uni (Invitrogen). Bacterial colonies containing the MCT1 ORF in the vector were identified in a PCR colony screen. A number of MCT1 positive colonies were grown, the plasmid DNA isolated and subjected to sequence analysis to ensure that no amino acid encoding mutations had been incorporated into the MCT1 ORF. The MCT1 ORF (BamHI/SalI) was then sub-cloned into the mammalian expression vector pcDNA3 (Invitrogen) and digested with BamHI/XhoI to generate the plasmid pcDNA3-hMCT1. The MCT1 ORF was then further subcloned into the insect expression vector pIZv5HIS (Invitrogen) and the S. cerevisiae expression plasmid, pACES14. Expression plasmids pcDNA3-hMCT1, pIZ-hMCT1, and pACES14-hMCT1 were purified and used to transform relevant host cells.

3.2 Rat MCT1

The open reading frame encoding rat MCT1 was amplified from a rat brain cDNA library (Origene) using the olIgonucleotide pair RM1-5′ (5′-TGCATGATCA-ATGCCACCTGCGATTGGCGGGCCAG-3′; SEQ ID No. 8) and RM1-3′ (5′-TGCAGCTAGCTCAG-ACTGGGCTCTCCTCCT-3′; SEQ ID No. 9) in a PCR. The resulting amplified DNA was digested with BclI/NheI and was ligated with pACES14 pre-digested with BamHI/NheI. The resulting insert DNA was sequenced to ensure that no mutations had been incorporated. Plasmid pACES14-rMCT1 was purified and used to transform relevant host cells.

3.3 Human MCT2

The open reading frame encoding human MCT2 was amplified from a full-length cDNA clone using the oligonucleotide pair MCT2-5′ (5′-AGC-TGG-ATC-CAC-CAT-GCC-ACC-AAT-GCC-AAG-3′; SEQ ID No. 10) and MCT2-3′ (5-GAC-TCT-CGA-GTT-AAA-TGT-TAG-TTT-CTC-TTT-CTG-A-3′; SEQ ID No. 11) in a PCR. The PCR fragment was subcloned into the vector pCR3.1 uni (Invitrogen). Bacterial colonies containing the MCT2 ORF in the vector were identified in a PCR colony screen. A number of MCT2 positive colonies were grown and the plasmid DNA isolated and subjected to sequence analysis to ensure that no amino acid-altering mutations had been incorporated into the MCT2 ORF. The full length open reading frame for human MCT2 was then subcloned into the S. cerevisiae expression plasmid, pACES14, the insect expression vector pIZv5HIS (Invitrogen), and the mammalian expression vector pcDNA3 (Invitrogen). Plasmids pACES14-hMCT2, pIZ-hMCT2, and pcDNA3-MCT2 were purified and used to transform relevant host cells.

3.4 Human MCT3

The human MCT3 ORF spans 4 exons in the human genome (Yoon et al., Genomics 60 (3), 366-370, 1999). Each of the four exons was amplified by PCR from human genomic DNA using the oligos MCT3-5′#3(5′-ATC-AGG-ATC-CAG-GCA-GCG-ATG-GGC-G-3′; SEQ ID No. 12)/MCT3-11# (5′-GAC-ACG-GGG-CCC-GTG-CCG-TAG-AGC-AT-3′; SEQ ID No. 13) for exon I; MCT3-10#(5′-CGG-CAC-GGG-CCC-CGT-GTC-CAG-CAT-3′; SEQ ID No.14)/MCT3-13# (5′-AGG-CCC-AGG-CCT-GTG-AGC-ACC-CCA-GC-3′; SEQ ID No. 15) for exon II; M3-G1(5′-GTT-CCC-GGA-TCT-GCT-GGG-TT-3′; SEQ ID No. 16)/M3-G2 (5′-TGG-AGC-TTC-CCT-GGG-TCT-AA-3′; SEQ ID No. 17) for exon III flanked by intron DNA; MCT3-14# (5′-CCC-TCT-GCC-GGC-CGC-CTG-GTG-GAT-GCG-TTG-AAG-3′; SEQ ID No. 18)/MCT3-3′#3 (5′-GTC-AAC-TAG-TCA-GAC-ACC-CAG-GGG-ATC-AAC-TGG-AG-3′; SEQ ID No. 19) for exon IV to ˜150 bp downstream of the termination codon. Exon III was then isolated the M3-G1/M3-G2 PCR product using the oligos MCT3-12# (5′-TGC-TCA-CAG-GCC-TGG-GCC-TGG-CCC-TCA-A-3′; SEQ ID No. 20)/MCT3-15# (5′-ACC-AGG-CGG-CCG-GCA-GAG-GGC-GGT-CC-3′; SEQ ID No. 21). A PCR product (I+II) was generated from the exon I and exon II PCR products using the oligos MCT3-5′#3/MCT3-13# and subcloned into pCRBluntII-TOPO to give pTOPOMCT3(I+II). A PCR product (III+IV) was generated from the exon III and exon IV PCR products using the oligos MCT3-12#/MCT3-3′#3 and subcloned into pCRII-TOPO to give pTOPOMCT3(III+IV). The MCT3 fragments from HindIII/StuI digested pTOPOMCT3(I+II) and from HindIII/StuI digested pTOPOMCT3(III+IV) were ligated to give the full length pTOPOMCT3 (in the pCRBluntII vector). A number of MCT3 positive colonies were grown and the plasmid DNA isolated and subjected to sequence analysis to ensure that no amino acid encoding mutations had been incorporated into the MCT1 ORF. Following sequence analysis, the full length open reading frame of human MCT3 was subcloned into the S. cerevisiae expression plasmid pACES14, the insect expression vector pIZv5HIS (Invitrogen), and the mammalian expression vector pcDNA3 (Invitrogen). Plasmids pACES14-hMCT3, pIZ-hMCT3 and pcDNA3-hMCT3 were purified and used to transform relevant host cells.

3.5 Human MCT4

A human dendritic cell cDNA clone (AC-DNA-1819) contains an incomplete copy of the MCT4 open reading frame. This was made full length by inserting ˜190 bp GBO212 (5′-TAG-GAA-GAA-GCC-CAA-AGA-GCC-ACA-G-3′; SEQ ID No. 22)/GBO213 (5′-GAC-TTC-TAG-AGC-CCA-GCC-ACT-CAG-ACA-CTT-GTT-TC-3′; SEQ ID No. 23) MCT4 3′ PCR fragment (amplified from a human naive T cell cDNA stock) on a NotI—XbaI fragment to make pGBAC41. The MCT4 ORF was subsequently amplified (using oligonucleotides GBO214 (5′-GAT-CGG-ATC-CAT-GGG-AGG-GGC-CGT-GGT-3′; SEQ ID No. 4)/GBO215 (5′-GTC-AGA-TAT-CGC-CAC-TCA-GAC-ACT-TG-3′; SEQ ID No. 25), which add BamHI and EcoRV restriction site recognition sequences to the 5′ and 3′ ends, respectively) from the full length copy and subcloned into the S. cerevisiae expression plasmid pACES14. The insert was sequenced to ensure that no mutations that would result in incorporation of altered amino acids had been incorporated during amplification and cloning. The human MCT4 ORF was then inserted into the insect expression vector pIZv5HIS (Invitrogen) and the mammalian expression vector pcDNA3. Plasmids pACES14-hMCT4, pIZ-hMCT4, and pcDNA3-hMCT4 were purified and used to transform relevant host cells.

3.6 Human MCT1/MCT2 Chimera

The human MCT1 and MCT2 cDNAs share a common recognition site for the restriction enzyme Hind III. This restriction site lies in the region that encodes the extracellular domain that separates the predicted transmembrane domains 5 and 6. The common Hind III site was used to create a human MCT1/MCT2 chimeric molecule, in the S. cervisiae expression plasmid pACES14, consisting of the amino terminus of MCT2 (TMs 1-5) and the carboxy terminus of MCT1 (TMs 6-12) (SEQ ID No: 40). Plasmid pACES14-hMCT2/MCT1 was purified and used to transform relevant host cells.

A series of further chimeric molecules was constructed by replacing parts of MCT1 with MCT2. Each of these bound ligand in the latter binding studies to greater or lesser extent.

### EXAMPLE 4

### Expression of MCT Proteins in Host Cells

The human breast cell line MDA-MB-231 had been previously identified as expressing low levels of MCT1 (Garcia et al., Cell. 76:865-873, 1994). The MDA-MB-231 cell line was grown. Filter binding assays (see Examples 6 and 7) carried out with the MDA-MB-231 cell line showed that the cells exhibited low level of binding to ³H-ligand C (˜6000 binding sites per cell). The cells were grown and transfected with pcDNA3-hMCT1. An increase in filter binding of ³H-ligand C was consistently measured for transiently transfected cells when compared with control cells transfected with empty vector.

The rat pancreatic β-cell line INS-1 has also been shown to exhibit low levels of lactate transport activity (Sekine et al., J. Biol. Chem. 269:4895-4902, 1994) and has been shown to express low levels of MCT proteins (Ishihara et al., J Clinical Invest. 104:1621-1629,1999; Zhao et al., Diabetes 50:361-366, 2001). INS-1 cells were grown and transfected with pcDNA3-hMCT1. An increase in filter binding of ³H-ligand C was consistently measured for transfected cells when compared with control cells transfected with empty vector.

Filter binding assays carried out with the insect cell line Sf9 showed that the cells exhibited low level of binding to ³H-ligand C. The Sf9 cell line was grown and transfected with pIZ-hMCT1. An increase in filter binding of ³H-ligand C was consistently measured for transfected cells when compared with control cells transfected with empty vector.

The yeast strain used for expression of MCTs was Saccharomyces cerevisiae Hansen BY4742 (Research Genetics) Mat alpha his3D1 leu2D0 lys2D0 ura3D0 Δjen:Kan^{r}. Yeast cells were made competent for DNA transformation and transformed with plasmid DNA using the Yeast Transformation Kit (SIGMA) according to the manufacturer's instructions. Expression of human MCTs was confirmed by Western analysis using the corresponding anti-human MCT C-terminal peptide antibody.

Antibody Generation:

Anti-human MCT1-4 C-terminal peptide antibodies were made by Cambridge Research Biochemicals. Peptide sequences used to immunise rabbits are as follows:

TABLE 4

| Human MCT | Peptide Sequence | SEQ ID No. |
|---|---|---|
| MCT1 | CQKDTEGGPKEEESPV | 26 |
| MCT2 | CKVSNAQSVTSERETNI | 27 |
| MCT3 | CTEPEIEARPRLAAESV | 28 |
| MCT4 | CEPEKNGEVVHTPETSV | 29 |

Three single amino acid variants of human MCT1 (Lys240Glu, Cys400Gly, Glu490Asp) were expressed in yeast cells, and membrane preparations containing these three variants were tested in filter binding assays. No alteration to compound binding was detected when compared to human MCT1 expressed in yeast. Single nucleotide changes that would result in the desired amino acid changes were incorporated into pACES-hMCT1 using the QuickChange Site-Directed Mutagenesis Kit (Stratagene). Incorporation of a mutated nucleotide residue that would result in an altered amino acid was confirmed by sequence analysis.

### EXAMPLE 5

### Cloning of a Strep Tag to the C-Terminus of MCT Proteins

The DNA sequence encoding the Strep Tag, AWRHPQFGG (SEQ ID No. 30) (Schmidt and Skera, 1993, Protein Engineering 6:109-122), was cloned and expressed at the C-terminus of hMCT1, hMCT2 and hMCT3.

MCT1

A three way ligation containing pACES14 (BamHI/NheI), the 5′ end of hMCT1 (BamHI/BspE1 digested pACES14-MCT1) and annealed oligonucleotides M1strp-1 (5′-CCG-GAC-CAG-AAA-GAC-ACA-GAA-GGA-GGG-CCC-AAG-GAG-GAG-GAA-AGT-CCA-GTC-GCT-TGG-AGA-CAT-CCA-CAA-TTT-GGT-GGT-TAA-T-3′; SEQ ID No. 31) and M1strp-2 (5′-CTA-GAT-TAA-CCA-CCA-AAT-TGT-GGA-TGT-CTC-CAA-GCG-ACT-GGA-CTT-TCC-TCC-TCC-TTG-GGC-CCT-CCT-TCT-GTG-TCT-TTC-TGG-T-3′; SEQ ID No. 32) encoding the C-terminus of MCT1 fused to the DNA encoding the strep-tag was carried out to generate the plasmid pACES14-MCT1-strep tag. Expression of the tagged material was confirmed by Western blotting using anti-hMCT1 Abs and a streptavidin-horseradish peroxidase conjugate (IBA GmbH). Ability of a yeast membrane preparation containing the strep tagged MCT1 to bind various radioligands (³H-ligand C, ¹²⁵I-ligand A) was tested and confirmed in the filter binding assay. Yeast membrane preparations containing the strep tagged MCT1 were then used to define conditions for an 384-well SPA assay using radioligand (¹²⁵I-ligand A) and streptavidin-coated PVT SPA beads (Amersham Pharmacia Biotech), as described below.

MCT2

A three way ligation containing pACES14 (BamHI/NheI), the 5′ end of hMCT2 (BamHI/DraIII digested pIZ-MCT2) and annealed oligonucleotides FM2strep2 (5′-GTG-TAA-CCT-CAG-AAA-GAG-AAA-CTA-ACA-TTG-CTT-GGA-GAC-ATC-CAC-AAT-TTG-GTG-GTT-AAT-3′; SEQ ID No. 33) and RM2strep2 (5′-CTA-GAT-TAA-CCA-CCA-AAT-TGT-GGA-TGT-CTC-CAA-GCA-ATG-TTA-GTT-TCT-CTT-TCT-GAG-GTT-ACACTCT-3′; SEQ ID No. 34) encoding the C-terminus of MCT2 fused to the DNA encoding the strep-tag, was carried out to generate the plasmid pACES14-MCT2-strep tag. Expression of the tagged material was monitored by Western blotting using anti-hMCT2 Abs and a streptavidin-horseradish peroxidase conjugate (IBA GmbH).

MCT3

The 3′ end of hMCT3 (from Topo-MCT3) was amplified in a PCR reaction with the primer pair Mct3E-2 (5′-GCCATCCTGCTGGTGAACTA-3′; SEQ ID No. 35) and M3-3st (5′-TAG-CTA-GTC-TAG-ATT-AAC-CAC-CAA-ATT-GTG-GAT-GTC-TCC-AAG-CTA-CAG-ACT-CGG-CAG-CCA-GCC-TCG-GCC-TCG-CC-3′; SEQ ID No. 36). The PCR fragment contains the 3′ end of hMCT3 fused to the DNA encoding the strep tag. The PCR product was digested with NotI and XbaI and placed in a three-way ligation with pACES14 BamHI/NheI and the 5′ end of hMCT3 (released from Topo-MCT3 digested with BamHI/NotI), to generate pACES14-hMCT3strep tag. Expression of the tagged hMCT3 was monitored by Western blotting using anti-hMCT3 Abs and a streptavidin-horseradish peroxidase conjugate (IBA GmbH). Ability of a yeast membrane preparation containing the strep tagged MCT3 to bind various radioligands (³H-ligand C, ¹²⁵I-ligand A) was tested in the filter binding assay.

### EXAMPLE 6

### Yeast Expressed MCT1 Filter Binding Assay

Assay to Measure the Potency of Selected Compounds Using a Filter Binding Assay

Competition assays can be used to measure the affinity of unlabelled compound for MCT1. Tritiated ligand C is included at a constant concentration and the compound to be tested is titrated. 10 μl of ³H-ligand C that had been diluted with assay buffer (50 mM HEPES, 0.1 mM EDTA, 0.15 M NaCl, pH 7.5, 0.5% BSA) was dispensed into wells of a polypropylene plate such that when the assay was made to 200 μl the final concentration would be 2.5 nM. 10 μl of compound in assay buffer was added to each well to give a final concentration typically covering the range 0.1 to 1000 nM. 180 μl of yeast membranes expressing MCT1 in assay buffer containing typically 0.5 to 1 μg total protein was added to start the reaction. Non-specific binding was measured in the presence of 1 μM unlabelled Ligand 1. The competition assay was incubated at 2 hours at room temperature with shaking. Experimental data points were usually carried out in triplicate. The membranes were harvested onto GF-B filter plates and washed with assay buffer without BSA, dried, scintillant added and counts detected using a tritium program on a Packard Top Count plate reader. The results were analysed by subtraction of the non-specific binding from each of the experimental points and then fitting a sigmoidal curve through a semi-log plot of the data in Microcal Origin. Calculated IC₅₀'s are shown in Table 5.

TABLE 5
Compound IC50s
calculated from the yeast membrane MCT1 filter binding assay.

| Ligand | IC₅₀ (nM) |
|---|---|
| 1 | 0.5 |
| 2 | 0.3 |
| 3 | 105 |
| 4 | 787 |

Key:

Ligand 1: (disclosed in WO 98/054190) 5-[(3-hydroxypropyl)thio]-3-methyl-1-(2-methylpropyl)-6-(1-napthalenylmethyl)thieno[2,3-d]pyrimidine-2,4(1H,3H)-dione

Ligand 1 appears in patent WO 98/054190 and has CAS number (Chem Abs Registry No) 216685-07-3.

Ligand 2: (disclosed in WO 99/029695) 2,6-dihydro-7-[(3-hydroxypropyl)thio]-2-methyl-4-(2-methylpropyl)-6-(1-napthanlenylmethyl)-1H-pyrrolo[3,4-d]pyridazin-1-one

Ligand 2 appears in patent WO 99/29695 and has CAS number 227321-12-2.

Ligand 3: (disclosed in WO 98/054190) 6-(4-quinolinylmethyl)--3-methyl-1-(2-methylpropyl)-thieno[2,3-d]pyrimidin-2,4(1H,3H)-dione

Ligand 4: (disclosed in WO 00/12514): 6-([benzothiazol-2-yl]methyl)-3-methyl-1-(2-methylpropyl)thieno[2,3-d]pyrimidine-2,4(1H,3H)-dione

### EXAMPLE 7

### MCT Filter Binding Assay

For yeast cells transformed with expression plasmids containing the human MCT 1-4 ORFs the ability of membrane preparations to bind radioligand was determined using a single ligand concentration/multi protein concentration binding assay. This generated an estimate of the amount of active binding protein present in each membrane preparation. The ligand concentration used will give an estimate of Bₘₐₓ that is approximately 90% of the true value. 10 μl of ligand A, B or C that had been diluted 50 fold with assay buffer (50 mM HEPES, 0.1 mM EDTA, 0.15 M NaCl, pH 7.5, 0.5% BSA) was dispensed into wells of a polypropylene microtitre plate. This gives a final radiolabelled ligand concentration of ˜2.5 nM. Non-specfic binding was measured in the presence of 1 μM Ligand 1. The membranes were diluted to give a known amount/180 μl assay buffer. This is typically in the 2000 nl to 0.2 nl range. For the membranes with high binding site number a more dilute membrane preparation should be added. 180 μl of each membrane solution was then added to each well and incubated at 2 hours at room temperature with shaking. The membranes were harvested onto GF-B filter plates and washed with assay buffer without BSA, dried, scintillant added and counts detected using the relevant program for each ligand on a Packard Top Count plate reader. Results from a typical experiment are shown in Table 6.

TABLE 6

| MCT (protein | Specific binding (cpm) | |
|---|---|---|
| quantity | Ligand C | Ligand A |
| assayed) | Average | Average |
| MCT1 (20 ug) | 5553 | 177453 |
| MCT1 (2 ug) | 3621 | 88841 |
| MCT2 (20 ug) | 213 | 14769 |
| MCT2 (2 ug) | 593 | 7286 |
| MCT3 (20 ug) | 40 | 0 |
| MCT3 (2 ug) | 0 | 0 |
| MCT4 (20 ug) | 0 | 872 |
| MCT4 (2 ug) | 81 | 405 |
| vector (20 ug) | 0 | 0 |
| vector (2 ug) | 0 | 1735 |

The filter binding assays of Examples 6 and 7 are suitable for high throughput screening of large compound libraries.

### EXAMPLE 8

### Yeast Expressed MCT1 Based SPA Assay

Scintillation Proximity Assay Method

| | |
|---|---|
| Final conditions: | 15 μg beads/well |
| | 3 μg yeast membranes/well |
| | 0.1 nM ¹²⁵I ligand A |
| | 50 mM HEPES pH 7.5, 0.1 mM EDTA, |
| | 150 mM NaCl + 0.05% BSA |

Streptavidin coated SPA beads were resuspended at 2.5 mg/ml in assay buffer (50 mM HEPES pH 7.5, 0.1 mM EDTA, 150 mM NaCl) and diluted to give a final concentration of 187 μg/ml in assay buffer without BSA. Yeast membranes expressing MCT1 with a streptavidin-binding sequence tag were then added to give a final concentration of 37 μg/ml protein and gently rolled for >30 minutes at room temperature. The beads/membranes were then washed by centrifugation at ˜650 g for 10 minutes and resuspended in assay buffer+0.05% BSA. The washing was repeated once before resuspended in the appropriate volume of assay buffer+0.05% BSA.

The iodinated ligand A was diluted from the stock into assay buffer+0.5% BSA to give a concentration of 1 nM. Non-specific binding was measured in the presence of Ligand 1 at 1 μM. Compound IC₅₀s were measured in triplicate over a 100,000-fold dilution range with individual dilutions at half log units. 10 μl of diluted ligand was added to each well, 10 μl of cold competitor ligand 1 was added to the non-specific control wells and 10 μl of buffer was added to the total counts control wells. Finally, 80 μl of the membrane/bead suspension was added to each well. The plates were sealed, incubated at room temperature for 3 hours, centrifuged at 650 g for 5 minutes before counting in a Packard Top Count plate reader using a protocol appropriate for ¹²⁵I. The raw counts were analysed by averaging replicates, subtraction of non-specific binding and the subsequent calculation of % inhibition of binding of the iodinated ligand. IC₅₀'s were calculated using Origin data fitting software and are shown in Table 7.

TABLE 7
Compound IC50s
calculated from the yeast membrane MCT1-strep tagged SPA assay.

| Ligand | IC₅₀(nM) |
|---|---|
| 1 | 0.6 |
| 2 | 0.5 |
| 3 | 15.4 |
| 4 | 32.2 |

This SPA assay is suitable for high throughput screening of large compound libraries.

### EXAMPLE 9

### Uptake of Labelled Lactate in Rat Red Blood Cells

Ligand 1 was dissolved at a concentration of 10 mM in DMSO and diluted in assay buffer (50 mM HEPES (pH 7.5), 1 mM EDTA, 50 mM NaCl) supplemented with 0.5% (w/v) BSA. 100× stock solutions were made for each concentration and 2 μl of these stocks diluted into 200 μl of rat blood to give the final compound concentration. The blood samples were incubated at room temperature for 2 hours. Lactate uptake was measured in each blood sample as follows: uptake was initiated by the addition of 50 μl of blood to 2 μl of ¹⁴C-lactate (12.5 μCi/ml; Amersham). The samples were incubated at room temperature for 30 s and then the reaction was halted by transfer of 20 μl of each sample onto 1 ml of ice-cold dibutyl-pthalate (Sigma). The red blood cells were separated from the plasma by centrifugation of the samples for 30 s at 15000 g in a bench-top microfuge. The supernatant was aspirated to waste taking care not to disturb the cell pellet. The cells were resuspended in 100 μl of 5% NP40 (v/v) in PBS and were transferred to a scintillation vial. 4 ml of scintillant were added and radioactivity was determined by scintillation counting. MCT1 inhibitors, such as Ligand 1, caused a dose-dependent decrease in the amount of [¹⁴C]-lactate uptake by rat red blood cells. Values from representative experiments were:

TABLE 8

| [¹⁴C]-Lactate | | | |
|---|---|---|---|
| uptake (cpm) | Experiment 1 | Experiment 2 | Experiment 3 |
| No Ligand 1 | 23000 ± 3000 | 22957 ± 1664 | 21497 ± 5304 |
| 1 μM Ligand 1 | 4362 ± 409 | 10426 ± 243 | 11146 ± 817 |

This method was also used to determine the effect of MCT1 inhibitors, including Ligand 1, on lactate uptake in human red blood cells and similar results were obtained.

### EXAMPLE 10

### Scintillation Proximity Assay (SPA) Using Jurkat T-Cell Membranes

Human Jurkat T cells were grown at 37° C. in RPMI 1640 medium supplemented with 5% foetal calf serum, 2 mM glutamine. Cells were harvested by centrifugation at 1500 g for 10 minutes. The cell pellets were washed twice with phosphate buffered saline (PBS) and centrifuged as above. The final cell pellet was resuspended in lysis buffer (50 mM HEPES (pH 7.8), 50 mM KCl, 10% glycerol, 0.1 mM EDTA, 1 mM DTT) and lysed by nitrogen cavitation. Unlysed cells and nuclei were removed by centrifugation at 1500 g for 10 minutes. The supernatant was then centrifuged at 100,000 g for 30 minutes at 4° C., and the pellet resuspended and homogenised in assay buffer (50 mM HEPES (pH 7.5), 1 mM EDTA, 150 mM NaCl). Aliquots of membrane preparation were stored at −80° C. until use.

Frozen Jurkat cell membranes were thawed on ice and then homogenised. Wheatgerm agglutinin-linked Scintillation Proximity Assay beads (Amersham) were rehydrated in assay buffer to a concentration of 100 mg/ml. 2 ml of Jurkat membranes were added per 0.5 ml (50 mg) of SPA beads and incubated overnight with constant agitation to allow the beads to coat with Jurkat membranes. The coated beads were then collected by centrifugation at 1500 g for 5 minutes and were washed twice in large volumes of assay buffer before being resuspended to a final concentration of 10 mg/ml in assay buffer. ¹²⁵I-ligand A was prepared as described above at a maximum specific activity of 2000 Ci mmol⁻¹. The radioligand was diluted in assay buffer containing 0.5% (w/v) BSA; the final concentration in the assay was approximately 0.1 nM. Assays were set up in 96-well flat-bottomed white opaque plates (Costar. Cat No: 3912). 10 μl of test compound and 10 μl of radioligand were incubated with 180 μl of SPA beads and membranes (0.04 mg beads). Non-specific binding was determined in the presence of 1 μM Ligand 1 and total binding was determined in the presence of vehicle alone. The plates were incubated for 3 hours at room temperature before quantitation of radioactivity proximal to the SPA beads by scintillation counting.

Compounds including Ligand 1 caused a dose-dependent reduction in the specific binding of ¹²⁵I-ligand A to Jurkat T-cell membranes. Values from a representative experiment were:

TABLE 9

| | ¹²⁵I-ligand A binding (cpm) |
|---|---|
| Total binding | 2490 ± 32 |
| Non-specific binding | 499 ± 20 |
| Specific binding (Total - Non-specific) | 1991 |

The mean Ki for Ligand 1 competition of ¹²⁵I-ligand A binding to Jurkat T-cell membranes was 0.074 nM (n=100).

### EXAMPLE 11

### Effect of MCT1 Inhibitors on T-Lymphocytes

Our studies have shown that the rate of lactate production by T-lymphocytes increases approximately 15-fold by 48 h after mitogenic stimulation (PMA/ionomycin). Western blotting analyses of stimulated PBMCs using Abs that recognise MCT1, MCT2 and MCT4 showed that these three MCTs are expressed 48 hours after stimulation. Compounds that bind to MCT1 with potencies in the region of 0.05-300 nM have been shown to cause a significant accumulation of intracellular lactate in the T-lymphocytes and a reduction in the amount of lactate in the extracellular medium. The potency of compound effects on lactate accumulation shows a significant correlation with inhibition of T-lymphocyte proliferation as measured by the rate of incorporation of [³H]thymidine into DNA. Data from compound activity in an SPA binding assay, 3-day lymphocyte proliferation assay and experiments on T-lymphocytes after 48 h of mitogenic stimulation (lactate levels and DNA synthesis) are shown.

Assay of T-Lymphocyte Proliferation

The cell signalling pathways triggered by T-cell activation through the T cell receptor and CD28 can be mimicked in vitro using a phorbol ester, phorbol 12-myristate 13-acetate (PMA), which activates protein kinase C, and ionomycin, which induces calcium release from internal cellular stores. PMA and ionomycin-stimulated proliferation of peripheral blood mononuclear cells (PBMC), of which the predominant cell type is T-lymphocytes, provides a suitable assay for measuring the ability of small molecules to block T-cell proliferation.

100 ml blood was collected by venopuncture of normal human volunteers into 3 tubes each containing 3 ml of 3.2% tri-sodium citrate solution. Blood was centrifuged at 850 g for 10 minutes and the plasma was removed. The cells were diluted to 50 ml with RPMI 1640 medium, and each 30 ml of diluted blood was layered over 20 ml Lymphoprep (Nycomed). The blood/Lymphoprep layers were centrifuged at 850 g for 20 minutes at 18° C. (with no brake). Cells at the interface were removed and were washed in RPMI 1640 by centrifugation at 850 g for 10 minutes. The cell pellets were then combined and were washed with 2×50 ml RPMI1640 at 680 g for 7 minutes. Cells were resuspended to a concentration of 1×10⁶ cells/ml in RPMI 1640 medium supplemented with 10% human AB serum (Quest Biomedical), L-glutamine (2 mM) and antibiotics (50 μg penicillin and streptomycin) (Complete medium).

Test compounds were dissolved in DMSO to give 10 mM stock solutions and were then diluted in complete medium to 20× the final assay concentration. 10 μl of compound in solution was then added to the 96-well flat bottom assay plate (final volume of 200 μl). Compounds were tested at a range of concentrations from 1×10⁻¹¹ M to 1×10⁻⁶ M. PMA and ionomycin were obtained from Sigma and made up to 1 mg/ml in DMSO. Both were diluted to 4× the final assay concentration in complete medium. 50 μl of each reagent was dispensed per well to give a final assay concentration of 0.5 ng/ml PMA and 500 ng/ml ionomycin. Control wells received 100 μl cells with PMA only (negative control 1), with ionomycin only (negative control 2) and 100 μl cells with 50 μl PMA and 50 μl ionomycin with no test compound (positive control). 100 μl complete medium was added to the negative controls. The plates were incubated at 37° C. for 72 h and the cultures were pulsed with ³H-thymidine (0.5 μCi/well; Amersham) for the final 6 hours. Cells were harvested on to glass fibre filter mats using a 96-well harvester (Tomtec inc., Orange, USA) and incorporated radioactivity was determined using a 1450 Microbeta counter (Perkin Elmer Life Sciences, Cambridge, England). MCT1 inhibitors, including Ligand 1, caused inhibition of T-cell proliferation with maximal inhibition of approximately 60%. IA₅₀ values were obtained from dose response curves using the 4-parameter logistic fit of a data analysis program.

IA₅₀ values are defined as the concentration of compound giving 50% of the maximum possible inhibition and were obtained from dose response curves using the 4-parameter logistic fit of a data analysis program.

Experiments on Activated T-Lymphocytes

PBMCs were prepared as described above by separation over Lymphoprep and T-lymphocytes were then enriched by purification on a nylon wool column. Briefly, 0.6 g nylon wool was inserted into a 10 ml syringe and this was autoclaved. The column was equilibrated with RPMI 1640 containing 20% human serum (HS) for 30 min at 37° C. The PBMC were resuspended in 1 ml of pre-warmed RPMI (20% HS) and were loaded onto the nylon wool column. The column was incubated for 45 min at 37° C. followed by elution of the T-cells by adding 10 ml RPMI (20% HS) (prewarmed to 37° C.) dropwise to the column. The T-cells obtained from the elution of the column were centrifuged (850 g for 5 min) and resuspended in RPMI containing L-glutamine and 5% human serum and stimulated with 0.5 ng/ml PMA and 500 ng/ml lonomycin for 48 h. T-cells were maintained in this growth medium at an initial cell density of 1×10⁶ cell per ml under standard cell culture conditions (37° C., 5% CO₂). After 48 h, the cells were harvested and prepared for the lactate and DNA synthesis assays by washing twice in RPMI 1640 and resuspending in fresh growth medium at 1×10⁶ cells per ml.

Assay of Lactate Levels by Lactate Oxidase Enzyme Activity

a) Intracellular lactate levels: Test compounds were dissolved in DMSO to give 10 mM stock solutions and were then diluted in complete medium to 10× the final assay concentration. 100 μl of each concentration were added to triplicate wells of a 24-well plate. T-cells were then added (1 ml per well) and were cultured for 4 h under standard cell culture conditions. The cells were then transferred from each well to microfuge tubes and were centrifuged at 360 g for 5 min at 4° C. The supernatant was discarded and the cell pellet was resuspended in 1 ml of ice-cold PBS, pH5.0. The cells were washed twice in PBS by centrifugation (360 g, 5 min, 4° C.) followed by resuspension of the cell pellet in 100 μl deionized water. The cells were incubated for 15 min at 4° C. to allow cell lysis to occur and the cell debris was removed by centrifugation at 15000 g for 10 min at 4° C. 10 μl of each supernatant sample were used for the lactate determination.

b) Extracellular lactate levels: Test compounds were dissolved in DMSO to give 10 mM stock solutions and were then diluted in complete medium to 10× the final assay concentration. 20 μl of each concentration were added to triplicate wells of a 96-well plate. T-cells were then added (200 μl per well, 2×10⁵ cells) and were cultured for 4 h under standard cell culture conditions. The cell supernatants were collected from each well after the cells were pelleted by centrifugation of the plate at 850 g for 5 min at 4° C. 10 μl of each supernatant sample were used for the lactate determination.

Lactate Reagent (Sigma, catalogue No. 735-10) was reconstituted according to the manufacturer's instructions. L-lactate (Sigma) was prepared as a 100 mM stock solution in PBS, pH 7.5 and was diluted in distilled water to give a standard curve in the range of 12.5-200 μM. The assay was carried out in 96-well plates at room temperature. 10 μl of standard or sample were added to each well followed by 200 μl of the Lactate Reagent. The plate was incubated for 15 min, and absorbance at 540 nm was then determined using a SPECTRAmaxPlus spectrophotometer (Molecular Devices). The data were collected and analysed using SOFTmax PRO software (Molecular Devices).

Assay of DNA Synthesis in Activated T-Lymphocytes

DNA synthesis was assessed by measuring the incorporation of [³H]-thymidine. The assay was carried out in a 96-well plate. 20 μl of test compounds at 10× the final concentration were added to the plate followed by 200 μl of T-cells per well (2×10⁵ cells). The cells were cultured for 4 h under standard cell culture conditions and cultures were pulsed with ³H-thymidine (0.5 μCi/well; Amersham) for the final hour of the incubation. Cells were harvested on to glass fibre filter mats using a 96-well harvester (Tomtec inc., Orange, USA) and incorporated radioactivity was determined using a 1450 Microbeta counter (Perkin Elmer Life Sciences, Cambridge, England).

Results

Compounds caused a dose-dependent decrease in the rate of [³H] thymidine incorporation and extracellular lactate concentration in 2 day activated T-lymphocytes. The maximum level of inhibition caused by Ligand 1 was 48.6±2.1% in the assay of [³H] thymidine incorporation and 30±1.9% inhibition in the assay of extracellular lactate. These values are representative of the maximum inhibition levels observed for the compounds shown below.

Ligand 1 caused a dose-dependent increase in the intracellular lactate concentration from below the limit of detection (6.25 nmoles per 10⁶ cells) to concentrations in the range of 26-37 nmoles per 10⁶ cells.

Mean±s.e.mean of pIA50 values (mean±range for n=2)

TABLE 10

| | | Extracellular | Intracellular | Proliferation |
|---|---|---|---|---|
| Ligand | DNA | Lactate | Lactate | (3 days) |
| 1 | 9.19 ± 0.03 | 9.16 ± 0.03 | 9.39 ± 0.07 | 9.46 ± 0.22 |
| | (n = 8) | (n = 8) | (n = 6) | (n = 28) |
| 2 | 8.87 ± 0.13 | 9.10 ± 0.17 | 9.26 ± 0.06 | 9.42 ± 0.29 |
| | (n = 3) | (n = 3) | (n = 3) | (n = 3) |
| 5 | 8.57 ± 0.21 | 8.42 ± 0.27 | 8.64 ± 0.21 | 8.78 ± 0.28 |
| | (n = 4) | (n = 4) | (n = 4) | (n = 10) |

Ligand 5: 6-[(4,5-Dichloro-2-methyl-1H-imidazol-1-yl)methyl]-1,2,3,4-tetrahydro-N-methoxy-N,3-dimethyl-1-(2-methylpropyl)-2,4-dioxo-thieno[2,3-d]pyrimidine-5-carboxamide Ligand 5 appears in International patent Application Number PCT/GB02/03250

### EXAMPLE 12

### Inhibition of B-Lymphocyte Proliferation

Splenic B-lymphocytes were obtained from Balb/c mice by disruption of the spleen through a nylon sieve. The resultant cellular suspension was washed three times by centrifugation. The spleen cells were then plated out in flat-bottomed 96 well microtitre plates at a concentration of 2-4×10⁵ cells per well. B-lymphocyte stimulators were added with or without compound. Stimulators employed were lipopolysaccharide (LPS) at 50 μg/ml, 8-mercaptoguanosine (8MG) at 100 μg/ml, or goat anti mouse IgM at 40 μg/ml. Ligand 2, a compound with MCT inhibitory activity, was added to the cultures in a concentration range of 10⁻¹⁰M to 10⁻⁵M. The cultures were incubated for 48 hours at 37° C. and pulsed with tritiated thymidine for the final 6 hours. The cells were harvested as described previously and thymidine uptake used as a measure of DNA synthesis.

Ligand 2 partially inhibited the proliferative response of the B-lymphocytes to LPS (IA₅₀=5×10⁻⁹M), and 8MG (IA₅₀=10⁻⁸M). Inhibition of the proliferative response to goat anti-mouse IgM was weak (IA₅₀=>10⁻⁷M).

### EXAMPLE 13

### In Vivo Activity of Compounds

Graft Versus Host Response

Compounds have been tested in the rat Graft versus Host Response (GVHR), which represents the immune elements associated with transplant rejection. The model was first described by Ford et al. (1970). The assay consisted of injecting a 100 ul volume 5×10⁷-1×10⁹ spleen cells from dark agouti (DA) rats into the right hind footpads of DA/Lewis F1 hybrid rats. A similar number of DA/Lewis spleen cells were injected into the right hind footpads to act as controls. The DA grafted cells were recognised by the recipient DA/Lewis rats as having “self” antigenic components whereas the DA graft cells recognised the “Le” elements of the F1 hybrids as being foreign and subsequently responded by a proliferative response. The increase in proliferation was measured by an increase of weight of the right lymph node compared to the control left lymph node. When the rats were dosed with compounds active in the MCT binding screen and the in vitro proliferation assays, these compounds were found to effectively inhibit the development of the GVHR.

Compounds were dosed either once or twice daily from the day of cellular challenge in the footpads until termination 7 days later. The most active compounds tested gave an ED50 of 1-3 mg/kg dosed by the subcutaneous route.

Inhibition of Murine Antibody Production

Balb/c mice were immunised with 0.5 mg/kg ovalbumin (OVA) and 200 mg/kg aluminium hydroxide gel in saline intraperitoneally and left for 29 days before being re-challenged intraperotoneally with 0.5 mg/kg ovalbumin in buffered saline vehicle and left for a further 10 days. Compound was dosed daily from re-challenge to termination prior to serum collection. Control groups were dosed with compound vehicle only. On completion of dosing, serum samples were taken from the mice and analysed for total and specific levels of IgE.

For total IgE, microtitre plates were coated with 5 ug/ml monoclonal rat anti-mouse IgE in phosphate buffered saline (PBS) and incubated overnight at 4° C. The plates were then washed four times with PBS containing 0.05% Tween 20 and then blocked with 1% BSA in PBS at room temperature for 2 hours. This was followed by two further plate washings.

The serum samples and IgE standards were added to the wells in duplicate and incubated overnight. The plates were then washed a further four times before adding 50 ul biotinylated monoclonal rat anti-mouse IgE appropriately diluted in 0.1% BSA in PBS for 2 hours. The plate was washed for a further four times before adding 50 ul streptavidin alkaline phosphatase conjugate appropriately diluted in 1% BSA in PBS at room temperature for 50 minutes. The plate was washed a further four times before enzyme substrate (paranitrophenyl phoshate in 1M diethanolamine buffer pH 9.8) at 1 mg/ml was added. Once the colour reaction developed, the plate was read at 405 nm. Levels of total IgE in serum samples were extrapolated from curve obtained with IgE standards.

For specific IgE, the methodology was similar except for the following. The microtitre plates were coated with 50 ug/ml rat anti-mouse IgE. The serum samples or normal control sera were added to the washed plates. Biotinylated OVA diluted in 0.1% BSA in PBS was used as enzyme marker.

The activity of the MCT inhibitor Ligand 1 dosed at 3 and 30 mg/kg by the subcutaneous route for 10 days following antigenic boost was 67% and 84% inhibition respectively for total IgE and 64% and 53% inhibition respectively for OVA specific IgE.

To measure the effect of the compound on IgG2a production, Balb/c mice were immunised with ovalbumin in poly I:C adjuvant (polyinosinic:polycytidylic acid adjuvant) and left for 14 days before being re-challenged with ovalbumin in buffered saline vehicle and left for a further 7 days. Serum samples were taken from the mice and analysed for total and specific IgG2a. Compound was dosed daily from re-challenge to termination prior to serum collection. For total IgG2A, microtitre plates were coated with 5 ug/ml of goat anti-mouse IgG2a in PBS and incubated overnight at 4° C. The plates were then washed four times with PBS containing 0.05% Tween 20 and then blocked with 1% BSA at room temperature for 2 hours. This was followed by two further plate washings. The serum samples and IgG2a standards were added to the wells in duplicate and incubated at 4° C. overnight. The plates were then washed a further four times before adding 50 ul alkaline phosphatase conjugated goat anti-mouse IgG appropriately diluted in 0.1% BSA in PBS and left at room temperature for an hour. The plate was washed a further four times before enzyme substrate (p-nitrophenyl phosphate in 1M diethanolamine buffer pH 9.8) at 1 mg/ml was added. Once the colour reaction developed, the absorbance of the wells at 405 nm was read in a spectrophotometer. Levels of total IgG2a in serum samples were extrapolated from the curve obtained with IgG2a standards.

For specific IgG2a, the methodology was similar except for following. The microtitre plates were coated with 50 ug/ml OVA. The serum samples or normal control sera were added to the washed plates. Alkaline phosphatase conjugated goat anti-mouse IgG2a was used as enzyme marker.

The activity of the MCT inhibitor Ligand 1 at 3 and 30 mg/kg dosed by the subcutaneous route for 7 days following antigenic boost on total and specific IgG2a production gave ED50's of 30 mg/kg and 10 mg/kg respectively.

### EXAMPLE 14

### Proliferation of the Human Erythroleukaemia Cell Line K562

K562 cells were cultured in RPMI1640 medium (Gibco) supplemented with 2 mM L-glutamine and 5% foetal calf serum (FCS)(complete medium). Ligand 1 was dissolved at a concentration of 10 mM in DMSO and was diluted in absolute ethanol to 20× the final concentration required in the assay. 10 μl of the diluted solutions were then added to the wells of a 96-well microtitre plate and the ethanol was allowed to evaporate. The K562 cells were diluted to 1×10⁵ cells per ml in complete medium and 100 μl was added to each well of the plate with the addition of a further 100 μl of complete medium to give a total volume of 200 μl. The plates were incubated for a total of 48 h at 37° C. in 5% CO₂, with the addition of AlamarBlue (Serotec) for the last 24 h. AlamarBlue is an oxidation-reduction indicator dye that monitors metabolic activity (high level of reduction) as a readout of cellular proliferation. Absorbance was measured at 600 nm (OD₆₀₀; oxidised form) and 570 nm (OD₅₇₀; reduced form) using a Spectromax spectrophotometer (Molecular Devices). The maximum level of proliferation (OD₅₇₀-OD₆₀₀) was determined in the absence of Ligand 1. Ligand 1 caused a significant reduction in OD₅₇₀-OD₆₀₀ with a mean pIA₅₀ of 8.7±0.2 (n=3).

### EXAMPLE 15

### Lactate Uptake Studies in Cells Expressing MCT Isoforms by Measurement of Changes in Intracellular pH

2′,7′-bis(carboxyethyl)-5(6)-carboxyfluorescein (BCECF) is a pH-sensitive dye that has been used by Wang et al. (Am. J. Physiol. 267 (Heart Circ. Physiol. 36): H1759-69, 1994) to measure changes in intracellular pH of single cells. As lactate entry into cells is proton-linked, the addition of exogenous lactate to cells expressing functionally active MCT isoforms results in a significant decrease in intracellular pH. Lactate uptake by this method can be measured in cells endogenously expressing MCT isoforms (e.g., K562 and MDA-MB231 cells) or in cells transfected with cloned MCT isoforms (e.g., MCT1, 3 and 4) expressed in SF9 or INS-1 cells as follows. The construction of plasmids comprised of human MCT1, 2, 3, and 4 inserted into the mammalian expression vector pCDNA3 is described in Example 3. INS-1 cells were transfected with pcDNA3-hMCT1 using the Fugene™ 6 Transfection Reagent (Roche Molecular Biochemicals, Indianapolis, Ind., USA) to manufacturers instructions. Stably transfected cells were selected with 100 μg/ml Genetecin (Gibco Laboratories, Grand Island, N.Y.) to generate INS-1 MCT1 mixed populations, which were subsequently dilution cloned to generate INS-1 MCT1 clones. The same procedure was performed to generate mixed populations and clones for pCDNA3-hMCT2, pCDNA3-hMCT3 and pCDNA3-hMCT4.

A 75 cm² flask of INS-1 cells (clones or mixed populations) transfected with either pCDNA3-hMCT1, pCDNA3-hMCT2, pCDNA3-hMCT3, pCDNA3-hMCT4 or pCDNA3.1 was washed with complete medium (RPMI1640 containing 10% FCS, 2 mM glutamine, 1 mM sodium pyruvate, 0.00035% β-mercaptoethanol) and the cells removed by incubation with 3 ml of accutase (Innovative Cell Technologies, La Jolla, Calif., USA.) for 5 mins at 37° C. 3 ml of complete medium was added to each flask and the cells removed by gentle agitation. The cell suspension was removed and centrifuged (465×g for 5 min). The resultant cell pellet was resuspended in 5 ml of RPMI 1640 media without serum. Cells were counted using a haemocytometer and resuspended in RPMI 1640 medium without serum at 1.0×10⁶ cells/ml. Cells were loaded with BCECF by addition of 1 μl of 1 mM stock solution per 1 ml of cells to give a final concentration of 1 μM. The BCECF was incubated with the cells for 30 min at room temperature in the dark. Following loading with BCECF, the cell suspensions were centrifuged (465×g for 5 min) and then washed twice in Tyrodes buffer (140 mM NaCl, 4 mM KCl, 0.2 mM CaCl₂, 1 mM MgCl₂, 10 mM HEPES, 10 mM glucose, pH 7.4). The BCECF-loaded cells were then resuspended at a concentration of 1.0×10⁷ cells/ml in Tyrodes buffer.

Following BCECF labelling, 10 μl of the relevant cell suspension was added to each well of a 96 well plate (Biocat Poly-D-Lysine coated clear-bottomed black plates, Becton Dickinson) and 10 μl of inhibitor or vehicle control was added per well at 10× final concentration. 90 μl of Tyrodes or pH calibration buffer (140 mM KCl, 1 mM MgCl₂, 20 mM HEPES, 1 mM EGTA, 0.01 mM Nigericin at pH values from 5.39 to 8.44) was added to each well. Plates were incubated for 60 min at room temperature in the dark. The 96 well plate was then centrifuged at 275 g for 5 mins to ensure that the cells formed a monolayer on the bottom of the plate. The 96 well plate was then placed in the FLEXstation and fluorescence was measured using the following wavelengths:

| Excitation | Emission | Cut off |
|---|---|---|
| Lm1 490 | 535 | 555 |
| Lm2 440 | 535 | 555 |

The experiment was set up so that a reading at time zero was made, the lactate was added to the plate, and then readings were made every 3 seconds for 3 mins. L(+)-lactate (Sigma) was prepared at 3 times the final concentration by dilution of a 1M lactate solution in Tyrodes buffer. 50 μl diluted lactate solution was then added to each well. The ratio of fluorescence at 490 nm/440 nm was calculated and used to prepare a pH calibration curve for wells containing the pH calibration buffer. The pH calibration curve was then used to determine the pH of test wells from the 490/440 nm fluorescence ratio.

The addition of exogenous lactate caused a significant reduction in the intracellular pH of INS-1 cells expressing human MCT1, 3 or 4. No change in intracellular pH in response to lactate addition was observed in untransfected INS-1 cells or in INS-1 cells expressing the pCDNA3.1 vector. 100 nM Ligand 2 (AR-C122982) completely abolished the decrease in intracellular pH observed on addition of exogenous lactate to INS-1 cells expressing human MCT1 (n=2).

Each of the publications referenced herein is hereby incorporated by reference in its entirety. To the extent that any definitions may conflict, the definitions set forth herein shall prevail.

1. A method for identifying a compound having therapeutic potential, the method comprising
(a) determining whether a test compound decreases monocarboxylate transport activity; and
(b) if the compound decreases said activity, identifying the compound as having therapeutic potential.

2. The method of claim 1, wherein the compound identified as having therapeutic potential is further tested in a cellular proliferation assay.

3. The method of claim 2, wherein the cellular proliferation assay tests whether the compound inhibits proliferation of activated T lymphocytes.

4. The method of claim 2, wherein the cellular proliferation assay tests whether the compound inhibits proliferation of cancer cells in vitro.

5. The method of claim 2, wherein the cellular proliferation assay tests whether the compound inhibits proliferation of cancer cells in vivo.

6. The method of claim 1, wherein the compound identified as having therapeutic potential is further tested in an in vivo or in vitro model of inflammation.

7. The method of claim 1, wherein the compound identified as having therapeutic potential is further tested in an in vivo or in vitro model of autoimmune disease or transplant rejection.

8. The method of claim 1, wherein step (a) comprises determining whether the compound inhibits the activity of a monocarboxylate transport protein.

9. The method of claim 8, wherein the protein is in a cell, a cell ghost, a cell membrane fraction, or a lipid vesicle.

10. The method of claim 1, wherein step (a) comprises determining whether the compound reduces the level of expression of a monocarboxylate transport protein in a cell.

11. The method of claim 1, wherein the monocarboxylate transport protein is a mammalian MCT1, 2, 3, or 4.

12. The method of claim 1, wherein the monocarboxylate transport protein is a human MCT1, 2, 3, or 4.

13. The method of claim 1, wherein the monocarboxylate transport protein is MCT1.

14. The method of claim 1, wherein as a result of the determination that the compound decreases monocarboxylate transport activity, the compound is identified as having therapeutic potential in the treatment of an immune-mediated disorder or cancer.

15. The method of claim 1, wherein the determining step comprises (i) providing a cell expressing the protein; (ii) contacting the cell with the test compound; and (iii) determining whether the test compound affects one or more of the following: monocarboxylate accumulation within the cell, monocarboxylate efflux from the cell, H+ efflux from the cell, or H+ accumulation within the cell; as an indication that the test compound inhibits the protein's monocarboxylate transport activity.

16. The method of claim 1, wherein the determining step comprises an assay selected from the group consisting of: rapid filtration of equilibrium binding mixtures, radioimmunoassays (RIA), fluorescence resonance energy transfer assays (FRET), scintillation proximity assay (SPA), measurement of intracellular pH, and the use of labelled substrates to measure transport.

17. A method for identifying a compound having therapeutic potential, the method comprising
(a) determining whether a test compound binds to a monocarboxylate transport protein; and
(b) if the compound binds to the protein, identifying the compound as having therapeutic potential.

18. The method of claim 17, wherein following step (b), the compound's ability to inhibit the activity of a monocarboxylate transport protein is tested.

19. The method of claim 17, wherein the determining step comprises ascertaining the binding affinity of the compound for the protein.

20. The method of claim 17, wherein the determining step comprises a competitive binding assay, using as competitive reagent a labelled second compound that specifically binds to the protein.

21. The method of claim 17, wherein the determining step comprises providing a cell expressing the protein, or a cell membrane preparation derived from the cell, and contacting the test compound with the cell or the preparation.

22. The method of claim 21, wherein the cell naturally expresses the protein.

23. The method of claim 21, wherein the protein is a recombinant protein expressed by the cell, the cell being transfected with a nucleic acid encoding the protein.

24. The method of claim 17, wherein the monocarboxylate transport protein is a mammalian MCT1, 2, 3, or 4.

25. The method of claim 17, wherein the monocarboxylate transport protein is a human MCT1, 2, 3, or 4.

26. The method of claim 17, wherein the monocarboxylate transport protein is MCT1.

27. A method for producing a therapeutic composition, the method comprising carrying out the method of claim 1 to identify a compound with therapeutic potential, and mixing the compound, or a derivative thereof, with a pharmaceutically acceptable carrier.

28. A method for producing a therapeutic composition, the method comprising carrying out the method of claim 17 to identify a compound with therapeutic potential, and mixing the compound, or a derivative thereof, with a pharmaceutically acceptable carrier.

29. A method of producing a therapeutic composition, the method comprising carrying out the method of claim 1 to identify a compound with therapeutic potential, and manufacturing a therapeutic composition comprising the compound in accordance with practices that ensure the sterility of the composition.

30. A method of producing a therapeutic composition, the method comprising carrying out the method of claim 17 to identify a compound with therapeutic potential, and manufacturing a therapeutic composition comprising the compound in accordance with practices that ensure the sterility of the composition.

31. The method of claim 29, wherein the composition is labelled either for use in a method of treating an immune-mediated condition or for use in a method of treating cancer.

32. The method of claim 30, wherein the composition is labelled either for use in a method of treating an immune-mediated condition or for use in a method of treating cancer.

33. A compound identified by the method of claim 1, provided that the compound is not within any of Formulae I-IX.

34. A compound identified by the method of claim 17, provided that the compound is not within any of Formulae I-IX.

35. A compound identified by the method of claim 1, wherein the compound is at least ten times as active against one of MCT1, 2, 3, or 4, as against any other of the four.

36. A compound identified by the method of claim 17, wherein the compound is at least ten times as active against one of MCT1, 2, 3, or 4, as against any other of the four.

37. A method of treating a human subject in need of treatment for a disease or condition characterised by T-cell activation or cellular proliferation, the method comprising administering to the subject a compound that inhibits cellular monocarboxylate transporter activity, other than a quinazolinedione compound or a compound of formulae I to IX.

38. A method of treating a human subject in need of treatment for an immune-mediated disorder or cancer, the method comprising administering to the subject a compound that inhibits cellular monocarboxylate transporter activity, provided that the compound is other than a quinazolinedione compound or a compound of formulae I to IX.

39. A method of treating a human subject in need of treatment for an immune-mediated disorder or cancer, the method comprising identifying a subject as being in need of said treatment, and administering to the subject a compound that inhibits a monocarboxylate transporter other than MCT1 or MCT2.

40. The method of claim 39, wherein the monocarboxylate transporter is MCT3 or MCT4.

41. A method of treating a patient suffering from or likely to suffer from an immune-mediated disorder or cancer, the method comprising (i) identifying a compound as being an inhibitor of monocarboxylate transport in a cell, and (ii) administering to the patient an effective amount of the compound.

42. The method of claim 41, wherein the compound is a broad spectrum inhibitor capable of potently inhibiting at least two monocarboxylate transport proteins.

43. The method of claim 41, wherein the compound is at least ten times as active against one of MCT1, 2, 3, and 4, as against any other of the four.

44. A method for treating a patient suffering from an immune-mediated disorder or cancer, the method comprising administering to said patient an effective amount of a compound that specifically reduces expression of an MCT, the compound being selected from the group consisting of an anti-sense molecule, a ribozyme molecule, an RNAi molecule and a triple helix forming molecule.

45. A method of inhibiting T-cell or B-cell proliferation in a human, the method comprising identifying a human in need of such inhibition, and administering to the human a compound capable of specifically inhibiting monocarboxylate transport within a T-cell or B-cell.
